(19) United States
(12) Patent Application Publication
(10) Pub. No.: US 2012/0157837 A1
(43) Pub. Date: Jun. 21, 2012

(54) ULTRASOUND PROBE AND ULTRASOUND EXAMINATION DEVICE USING THE SAME

(76) Inventors: Takayuki NAGATA, Osaka (JP); Shinichi Kadowaki, Fukuoka (JP); Hiroyuki Furuya, Fukuoka (JP)

(21) Appl. No.: 13/407,920

(22) Filed: Feb. 29, 2012

Related U.S. Application Data
(63) Continuation of application No. PCT/JP2011/000508, filed on Jan. 31, 2011.

(30) Foreign Application Priority Data
Feb. 1, 2010 (JP) 2010-020036
Mar. 25, 2010 (JP) 2010-070764
Mar. 25, 2010 (JP) 2010-070766

(51) Int. Cl. A61B 8/00

(52) U.S. Cl. 600/437

(57) ABSTRACT

An ultrasound probe which is capable of structurally improving resolution is provided. This ultrasound probe includes: an ultrasound probe which transmits ultrasound waves to a subject; and an optical probe which detects, with use of light, the ultrasound waves reflected off internal tissue of the subject, and openings of the ultrasound probe are larger than openings of the optical probe. With this, reflected waves (ultrasound waves) from the subject can be detected by the large openings, so that high resolution can be obtained relative to a width of the transmitted ultrasound waves.

### CROSS REFERENCE TO RELATED APPLICATIONS

This is a continuation application of PCT Patent Application No. PCT/JP2011/000508 filed on Jan. 31, 2011, designating the United States of America, which is based on and claims priority of Japanese Patent Applications No. 2010-020036 filed on Feb. 1, 2010, No. 2010-070766 filed on Mar. 25, 2010, and No. 2010-070764 filed on Mar. 25, 2010. The entire disclosures of the above-identified applications, including the specifications, drawings and claims are incorporated herein by reference in their entirety.

### BACKGROUND OF THE INVENTION

(1) Field of the Invention

The present invention relates to ultrasound probes and ultrasound examination devices using the same and particularly relates to an ultrasound probe which receives or transmits and receives ultrasound waves to generate an ultrasound image and relates to an ultrasound examination device using the ultrasound probe.

(2) Description of the Related Art

The ultrasound examination device is a device which transmits ultrasound waves to an inside of a subject and forms an image representing information on the inside of the subject based on waves reflected inside the subject. The ultrasound examination device described as above has features, such as real-time operability, convenience, and non-invasiveness, and therefore has also been used to monitor pulsations of hearts, conditions of fetuses, and the like in applications where a subject is a living body. Furthermore, in recent years, the ultrasound examination device has been increasingly introduced to detect breast cancer and the like and therefore is required to have still higher spatial resolution.

The spatial resolution of the ultrasound examination device is classified into distance resolution in the depth direction and azimuth resolution in the azimuth direction. The distance resolution depends on a pulse length of a transmitted beam and becomes better as the number of waves included in pulse waves decreases and as the frequency of ultrasound waves increases. This distance resolution can be as high as the resolution of computer tomography (CT). On the other hand, the azimuth resolution depends on a width of a transmitted or received ultrasound beam in the azimuth direction (which width will be hereinafter referred to as a beam width) and therefore has had difficulty being high in a wide range, both at a short distance and at a long distance.

Accordingly, an ultrasound examination device which requires high spatial resolution in a wide range increasingly tends to use an array-type ultrasound probe that is capable of controlling a focal position of an ultrasound beam to be transmitted or received. This array-type ultrasound probe includes a plurality of transducers arranged in one dimension or in two dimensions and controls a focal position of a beam to be transmitted or received, by controlling the timing of voltage application to each of the transducers.

As an example of a method of improving the spatial resolution in the case of using the ultrasound probe described as above, there is what is called a multistage focus method (for example, Patent Literature 1: Japanese Unexamined Patent Application Publication No. 2002-058671) in which a beam is transmitted plural times, each with a different focal position in the depth direction, and signals of waves reflected from around the respective focal positions of the transmitted beams are synthesized. Furthermore, what is called a dynamic focus method (for example, Patent Literature 2: Japanese Unexamined Patent Application Publication No. 2004-313485) has been in practical use, in which signals are received each with a focal position of a received beam different in the depth direction. In addition, with the improvement of semiconductor technology, a method using digital beam forming (for example, Patent Literature 3: Japanese Unexamined Patent Application Publication No. 2001-276058) has as become available in which received signals are temporarily stored in a digital memory and according to a desired focal position, the received signal is read out and subject to phasing addition.

However, out of the array-type ultrasound probes described as above, especially for the two-dimensional array probe, the microfabrication of a piezoelectric element included in the transducer and the electrical connections to a large number of piezoelectric elements are technically difficult.

As its solution, a sensor designed to convert an ultrasound signal into a light signal and detect it has been proposed as a method which eliminates the need of the electrical connections to the large number of piezoelectric elements, and a method using a fiber bragg grating and a method using a Fabry-Perot resonator structure have been reported. Furthermore, a method using an optical detection type ultrasound sensor having a two-dimensional detector plane has also been proposed (for example, Patent Literature 4: Japanese Unexamined Patent Application Publication 2004-000482).

### SUMMARY OF THE INVENTION

However, the above conventional techniques are not satisfactory in terms of improving the azimuth resolution of the ultrasound examination device and furthermore have respective problems, as described below.

For example, in the structure disclosed by Patent Literature 1, it is necessary to transmit and receive ultrasound waves plural times with different focal depths in the same direction, which imposes a problem of an increase in a length of shooting time to achieve high resolution in a wide range. In contrast, with the structures disclosed by Patent Literatures 2 and 3, it is possible to set a plurality of focal points of beams to be received, per one transmission, so as to increase the resolution, which allows an improvement in the resolution in a wide range without increasing the shooting time.

However, each of the methods disclosed by Patent Literatures 1 to 3 is a method which improves the resolution by a signal detection method and through processing on detected signals, which means that they do not structurally improve the resolution. It can however be inferred that a combination of the above methods and a structure which structurally achieves high resolution will result in still higher resolution.

Furthermore, the structure disclosed by Patent Literature 4 makes it possible to provide a two-dimensional array probe by converting a received signal into a light signal and detecting it to eliminate the need of the microfabrication of the piezoelectric elements and the electrical connections thereto. However, only the signal detection method of the receiving device, especially, the improvement on the S/N ratio, has been studied while no studies have been made on the resolution.

The present invention has been devised in view of the above-described circumstances and has an object to provide an ultrasound probe capable of structurally improving resolution and to provide an ultrasound examination device using the ultrasound probe.

In order to achieve the above object, an ultrasound probe according to an aspect of the present invention includes: an ultrasound transmitting unit configured to transmit ultrasound waves to a subject; and an ultrasound detecting unit configured to detect, using light, the ultrasound waves reflected off internal tissue of the subject, wherein an area of a region, of the ultrasound detecting unit, for detecting the ultrasound waves reflected off the internal tissue of the subject is larger than an area of a region, of the ultrasound transmitting unit, for transmitting the ultrasound waves to the subject.

With this structure, waves (ultrasound waves) reflected from a subject can be detected through large openings, which leads to high resolution as compared to a width of transmitted ultrasonic waves. Furthermore, not only the width of ultrasonic waves to be transmitted but also a scanning pitch can be increased, with the result that the number of times to transmit and receive the ultrasound waves in scanning can be smaller, which allows a decrease in the length of shooting time.

Furthermore, by detecting ultrasound echoes with use of light, the ultrasound detecting unit no longer requires electrical connections and can have a structure in which a large number of receiving spots are easily arranged in two dimensions.

With this, it is possible to provide an ultrasound probe which is capable of structurally improving resolution.

Here, it may be possible that the ultrasound detecting unit includes: a light source; an optical system that irradiates the subject with light emitted by the light source; and a light-receiving element that receives reflected light from the subject and detects a signal corresponding to the reflected light, and the ultrasound detecting unit is configured to detect vibration of a surface of the subject from a change in the signal detected by the light-receiving element, to detect the ultrasound waves reflected off the internal tissue of the subject, the vibration being caused by the ultrasound waves propagated to the surface of the subject.

This structure enables a structure in which ultrasound waves can be detected with use of light, with the result that the electrical connections are no longer necessary, which makes it easy to increase the number and area of spots.

Furthermore, it may be possible that the ultrasound detecting unit further includes a reflector disposed on the subject so as to be in close contact with the subject, the reflector reflecting the light with which the subject has been irradiated, according to the vibration of the surface of the subject, the vibration being caused by the ultrasound waves reflected off the internal tissue of the subject, and the ultrasound detecting unit is configured to receive, by the light-receiving element, the light reflected by the reflector, as the reflected light, to detect the ultrasound waves reflected off the internal tissue of the subject.

With this, the light emitted from the light source is directed to the reflector, which means that the subject is not directly irradiated with the light emitted from the light source and therefore can be kept safe. In addition, since the light emitted from the light source is reflected by the reflector, it is possible to reduce attenuation of the emitted light and thereby obtain a sufficient amount of light.

Furthermore, it may be possible that the ultrasound detecting unit includes a light-splitting element that splits, into a first light for examination and a second light for reference, the light emitted by the light source, the optical system emits, to the subject, the first light resulting from the splitting of the light-splitting element, and multiplexes the first light reflected off the subject with the second light resulting from the splitting of the light-splitting element, to generate multiplexed light so that the light-receiving element receives the multiplexed light as the reflected light, the multiplexed reflected light has a beat frequency corresponding to a difference in optical frequency between the reflected first light and the second light and is frequency-modulated according to the vibration of the surface of the subject, the vibration being caused by the ultrasound waves propagated to the surface of the subject, and the ultrasound detecting unit is configured to detect the vibration of the surface of the subject from a change in a signal obtained by the frequency-modulation of the multiplexed reflected light detected by the light-receiving element, to detect the ultrasound waves reflected off the internal tissue of the subject, the vibration being caused by the ultrasound waves propagated to the surface of the subject.

With this structure, the optical system can be a heterodyne interference optical system, which allows the ultrasound waves reflected off the internal tissue of the subject to be detected by detecting coherent light.

Here, it may be possible that the light-splitting element is composed of a semi-transmissive element that transmits, as the first light, part of the light emitted by the light source, and reflects, as the second light, the other part of the light emitted by the light source.

With this structure, the light can be separated into reference light and detection light at a position near the subject, which produces an effect of reducing vibration (body motion) noise.

Furthermore, it may be possible that the reflector has a plurality of regions and includes: an ultrasound converging unit formed in close contact with the subject and configured to converge, for each of the regions, ultrasound waves indicating the vibration of as the surface of the subject; and a first reflector element formed in a surface of the ultrasound converging unit, the surface being different from a surface in close contact with the subject, the first reflector element reflecting the light with which the subject has been irradiated, the ultrasound converging unit is configured to converge and amplify, for each of the regions, the ultrasound waves indicating the vibration of the surface of the subject, and propagate, to the first reflector element, the ultrasound waves resulting from the converging and the amplification, and the first reflector element reflects the light with which the subject has been irradiated, with reflectance which varies depending on the amplified ultrasound waves.

This structure enables a Fabry-Perot resonator structure, which allows the ultrasound waves reflected off the internal tissue of the subject to be detected by detecting a change in reflectance in this resonator structure. Furthermore, with this resonator structure, it is possible to improve receiving sensitivity by amplifying the ultrasound waves reflected off the internal tissue of the subject.

Here, for example, it may be possible that the first reflector element includes: a first multilayer film and a second multilayer film each formed by alternately stacking films having different refractive indices; a slit for dividing the second multilayer film into the detection regions; and a spacer formed between the first multilayer film and the second multilayer film, the spacer fixing one end of each of second multilayer films resulting from the division of the second multilayer film by the slit, the spacer forms a space between the first multilayer film and the second multilayer film, and the first reflector element propagates the ultrasound waves converged by the ultrasound converging unit, to an area near the other unfixed end of each of the second multilayer films resulting from the division so that the second multilayer film is deformed, and the propagated ultrasound waves are amplified. Furthermore, it may be possible that the first reflector element includes: a first multilayer film and a second multilayer film each formed by alternately stacking films having different refractive indices; a slit for dividing the second multilayer film into the regions; and a spacer formed between the first multilayer film and the second multilayer film, the spacer fixing one end of at least every other one of second multilayer films resulting from the division of the second multilayer film by the slit, and the first reflector element propagates the ultrasound waves converged by the ultrasound converging unit, to a center or a center of gravity of each of the second multilayer films so that the second multilayer film is deformed, and the propagated ultrasound waves are amplified. Furthermore, it may be possible that the light source emits the light of a narrow wavelength band, the first reflector element includes: a first multilayer film and a second multilayer film which are each formed by alternately stacking films having different refractive indices and have substantially identical reflection properties; and a substrate on which the first multilayer film is formed and illuminating light emitted from the light source is incident, the first multilayer film is disposed opposite to the second multilayer film so as to form a resonator structure, and the first reflector element propagates the ultrasound waves converged by the ultrasound converging unit, to vary a resonator length of the resonator structure and vary an amount of reflected light originated from the light emitted to the subject.

At this time, the ultrasound detecting unit is configured to adjust a wavelength of the light to be emitted from the light source, so as to minimize reflectance in the first reflector element, and then detect, using the light, the ultrasound waves reflected off the internal tissue of the subject.

With this structure, it is possible to improve a change of rate in reflectance at the time of detecting the ultrasound waves with use of light and thereby improve the receiving sensitivity.

Furthermore, in order to achieve the above object, an ultrasound examination device according to an aspect of the present invention includes the above ultrasound probe according to an aspect of the present invention.

Furthermore, it may be possible that an examining unit including a part of the ultrasound transmitting unit and the ultrasound detecting unit and configured to be used in close contact with the subject; and a main body including at least the other part of the ultrasound transmitting unit and the ultrasound detecting unit, wherein the main body includes at least the light source, the light-receiving element, and a part of the optical system.

Furthermore, it may be possible that a control unit is further included which is configured to control timing of the transmission of the ultrasound waves from the ultrasound transmitting unit, and the control unit is configured to control an amount of light to be emitted from the light source, according to an elapsed time from the transmission of the ultrasound waves from the ultrasound transmitting unit.

With this structure, it is possible to control a gain by adjusting an amount of light to be emitted, which produces an effect of simplifying a circuit included in the ultrasound examination device.

According to the present invention, it is possible to provide an ultrasound probe which is capable of structurally improving resolution and to provide an ultrasound examination device using the ultrasound probe. Specifically, the ultrasound probe and the ultrasound examination device using the ultrasound probe according to an aspect of the present invention produce an effect of providing high resolution and reducing the length of shooting time.

### BRIEF DESCRIPTION OF THE DRAWINGS

These and other objects, advantages and features of the invention will become apparent from the following description thereof taken in conjunction with the accompanying drawings that illustrate a specific embodiment of the present invention. In the Drawings:

FIG. 1A shows a schematic configuration of an ultrasound examination device in Embodiment 1 of the present invention;

FIG. 1B shows a schematic configuration of the ultrasound examination device in Embodiment 1 of the present invention;

FIG. 2 shows a specific configuration of an optical probe in Embodiment 1 of the present invention;

FIG. 3 illustrates signal waveforms of reference light and detection light which are detected by a light detector in Embodiment 1 of the present invention;

FIG. 4A illustrates an output waveform of the light detector in Embodiment 1 of the present invention;

FIG. 4B illustrates an output waveform of the light detector in Embodiment 1 of the present invention;

FIG. 5A illustrates resolution of the ultrasound examination device in Embodiment 1 of the present invention;

FIG. 5B illustrates resolution of the ultrasound examination device in Embodiment 1 of the present invention;

FIG. 6 shows a specific configuration of the optical probe in Embodiment 1 of the present invention;

FIG. 7 shows another specific configuration of the optical probe in Embodiment 1 of the present invention;

FIG. 8 shows another configuration of the probe for examination in the ultrasound examination device in Embodiment 1 of the present invention;

FIG. 9 shows a variation of the specific configuration of the optical probe in Embodiment 1 of the present invention;

FIG. 10A shows another specific configuration of the optical probe in Embodiment 1 of the present invention;

FIG. 10B shows another specific configuration of the optical probe in Embodiment 1 of the present invention;

FIG. 11A shows configurations of an optical probe and a receiving unit in Embodiment 2 of the present invention;

FIG. 11B shows specific configurations of the optical probe and the receiving unit in Embodiment 2 of the present invention;

FIG. 12A schematically shows a configuration of an ultrasound detection element in Embodiment 2 of the present invention;

FIG. 12B shows spectroscopic properties of the ultrasound detection element shown in FIG. 12A;

FIG. 13A illustrates a method of manufacturing the ultrasound detection element in Embodiment 2 of the present invention;

FIG. 13B illustrates a method of manufacturing the ultrasound detection element in Embodiment 2 of the present invention;

FIG. 13C illustrates a method of manufacturing the ultrasound detection element in Embodiment 2 of the present invention;

FIG. 13D illustrates a method of manufacturing the ultrasound detection element in Embodiment 2 of the present invention;

FIG. 14A illustrates a variation of a configuration of an ultrasound detection element in Embodiment 3 of the present invention;

FIG. 14B illustrates a variation of the configuration of the ultrasound detection element in Embodiment 3 of the present invention;

FIG. 15A illustrates another variation of the configuration of the ultrasound detection element in Embodiment 3 of the present invention;

FIG. 15B illustrates another variation of the configuration of the ultrasound detection element in Embodiment 3 of the present invention;

FIG. 16A shows configurations of an optical probe and a receiving unit in Variation 1 of Embodiment 3 of the present invention;

FIG. 16B shows configurations of the optical probe and the receiving unit in Variation 1 of Embodiment 3 of the present invention;

FIG. 17 schematically shows a configuration of an ultrasound detection element in Variation 2 of Embodiment 3 of the present invention;

FIG. 18A schematically shows a configuration in which a transducer group and a detection element group are arranged in Embodiment 4 of the present invention;

FIG. 18B schematically shows a configuration in which the transducer group and the detection element group are arranged in Embodiment 4 of the present invention;

FIG. 19A schematically shows another configuration in which the transducer group and the detection element group are arranged in Embodiment 4 of the present invention;

FIG. 19B schematically shows another configuration in which the transducer group and the detection element group are arranged in Embodiment 4 of the present invention;

FIG. 20A shows a configuration of an ultrasound examination device in Variation 1 of Embodiment 4 of the present invention;

FIG. 20B shows a configuration of the ultrasound examination device in Variation 1 of Embodiment 4 of the present invention;

FIG. 21A shows a configuration of an ultrasound examination device in Variation 2 of Embodiment 4 of the present invention; and

FIG. 21B shows a configuration of the ultrasound examination device in Variation 2 of Embodiment 4 of the present invention.

### DESCRIPTION OF THE PREFERRED EMBODIMENTS

Embodiments of the present invention are described below with reference to the drawings. The same constituents are denoted with the same signs and descriptions of the same constituents may not be repeated. Furthermore, the drawings schematically mainly show respective constituents to facilitate understanding and therefore may not be precise in terms of shapes and the like.

### Embodiment 1

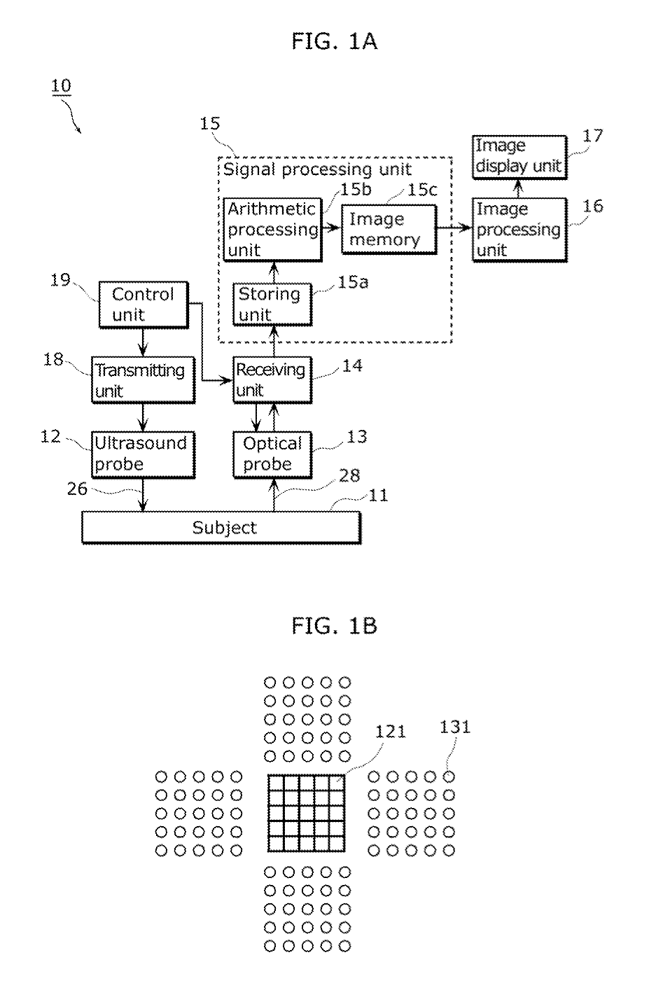

FIGS. 1A and 1B show a schematic configuration of an ultrasound examination device 10 in Embodiment 1 of the present invention. FIG. 1A is a block diagram showing the configuration, and FIG. 1B shows arrangement of points that transmit and receive ultrasound waves in a probe for examination in the ultrasound examination device 10.

The ultrasound examination device 10 includes: an ultrasound probe 12 that transmits the ultrasound waves to a subject 11; an optical probe 13 that detects micro vibration of a surface of the subject 11 with use of light, modulates such information, and outputs the resultant information; a receiving unit 14 that amplifies a detection signal obtained by demodulating an output signal of the optical probe 13, applies digital conversion to the detection signal, and outputs the resultant signal; a signal processing unit 15 that performs phasing addition, such as digital beam forming, using the signal outputted from the receiving unit 14; an image processing unit that performs processing of, for example, rendering a three-dimensional image, based on three-dimensional data generated by the signal processing unit 15; an image display unit 17 that displays an image based on the resultant image data; a transmitting unit 18 that generates a drive signal for the ultrasound probe 12 to transmit the ultrasound waves; and a control unit 19 that controls the transmitting unit 18 so that the transmitting unit 18 generates the drive signal with predetermined timing. Furthermore, the signal processing unit 15 includes: a storing unit 15a in which the detection signal outputted from the receiving unit 14 is stored; an arithmetic processing unit 15b that performs beam forming processing based on signal data stored in the storing unit 15a; and an image memory 15c in which information obtained in the arithmetic processing unit 15b is stored as three-dimensional data.

As shown in FIG. 1B, the probe for examination in the ultrasound examination device 10 is configured such that receiving spots 131 of the optical probe 13 that receive the ultrasound waves are located around transducers 121 of the ultrasound probe 12 that transmit the ultrasound waves. It is to be noted that the configuration shown in FIG. 1B is an example, and another arrangement is applicable when the ultrasound probe 12 and the optical probe 13 are configured as separate units so that openings of the transmitting unit are larger than openings of the receiving unit. Furthermore, the transducers 121 and each set of the receiving spots 131 do not need to be arranged in-plane and may be arranged along a contour of a subject as long as their relative positional relation is clear. It is to be further noted that the ultrasound probe 12 and the optical prove 13 are configured as separate units, but are provided in a single housing as the probe for examination in the ultrasound examination device 10. Thus, as shown in FIG. 1B, points that transmit and receive the ultrasound waves are arranged as a configuration of the probe for examination. In other words, the probe for examination in the ultrasound examination device 10 includes the ultrasound probe 12 and the optical probe 13 as separate units with the two-dimensional arrangement of the points that transmit and receive the ultrasound waves.

More specifically, it is sufficient that the probe for examination includes a configuration of the ultrasound probe 12 which has at least a function of transmitting the ultrasound waves, and a configuration of the optical probe 13 which has at least a function of receiving, in form of light information, the micro vibration of the surface of the subject 11. The ultrasound examination device 10 therefore includes the probe for examination and a device main body (on a diagnostic device side) including a configuration other than the probe for examination. Specifically, the device main body includes a component part, out of the ultrasound probe 12 and the optical probe 13, which is not included in the probe for examination, the receiving unit 14, the signal processing unit 15, the image processing unit 16, the image display unit 17, the transmitting unit 18, and the control unit 19.

The ultrasound probe 12 corresponds to an ultrasound transmitting unit in an implementation of the present invention and transmits the ultrasound waves to the subject. Specifically, the ultrasound probe 12 is composed of a transducer array in which a plurality of transducers are arranged in two dimensions and each of the transducers is constituted by forming an electrode on a piezoelectric element made of, for example, piezoelectric ceramic represented by lead zirconate titanate (PZT). The transducer array is configured to not only generate ultrasound pulses but also focus and deflect the generated ultrasound waves by applying, to the electrode of each of the transducers, pulsed voltage processed with a delay and transmitted from the transmitting unit 18. This structure allows the ultrasound probe 12 to transmit ultrasound waves 26 in three-dimensional directions to perform sector scanning. The ultrasound probe 12 may be composed of a probe of a mechanical swing type, which enables three-dimensional scanning.

The optical probe 13 corresponds to an ultrasound detecting unit in an implementation of the present invention and detects, with use of light, the ultrasound waves (ultrasound echoes 28) reflected off the internal tissue of the subject. Here, an area of a region (openings), of the optical probe 13, for detecting the ultrasound waves reflected off the internal tissue of the subject 11 is larger than an area of a region (openings), of the ultrasound probe 12, for transmitting the ultrasound waves to the subject 11. Furthermore, the optical probe 13 includes: a light source; an optical system which irradiates the subject 11 with light emitted by the light source; and a light-receiving element which receives light reflected on the subject 11 and detects a signal according to the reflected light. From a change in the signal detected by the light-receiving element, the optical probe 13 detects vibration of a surface of the subject 11 caused by the ultrasound waves propagated to the surface of the subject 11, to detect the ultrasound waves (the ultrasound echoes 28) reflected off internal tissue of the subject 11.

In other words, the optical probe 13 is composed of, for example, a heterodyne interference optical system or a Fabry-Perot resonator structure and is typically composed of the heterodyne interference optical system. The following is described on the assumption that the optical probe 13 is composed of the heterodyne interference optical system. Specifically, the optical probe 13 is configured to detect the ultrasound waves by separating frequency-modulated laser light into detection light and reference light so that the detection light is emitted to the ultrasound detection element located on the surface of the subject 11 and the detection light reflected by the ultrasound detection element interferes with the reference light. Furthermore, the optical probe 13 provides a detected signal (an output signal) to the receiving unit 14. This output signal has a waveform resulting from frequency modulation (FM) of carrier waves.

This output signal is then demodulated by the receiving unit 14, thereby resulting in the micro vibration of the surface of the subject 11, that is, ultrasound echo information. A specific configuration of this optical probe 13 and this signal detection principle will be described below.

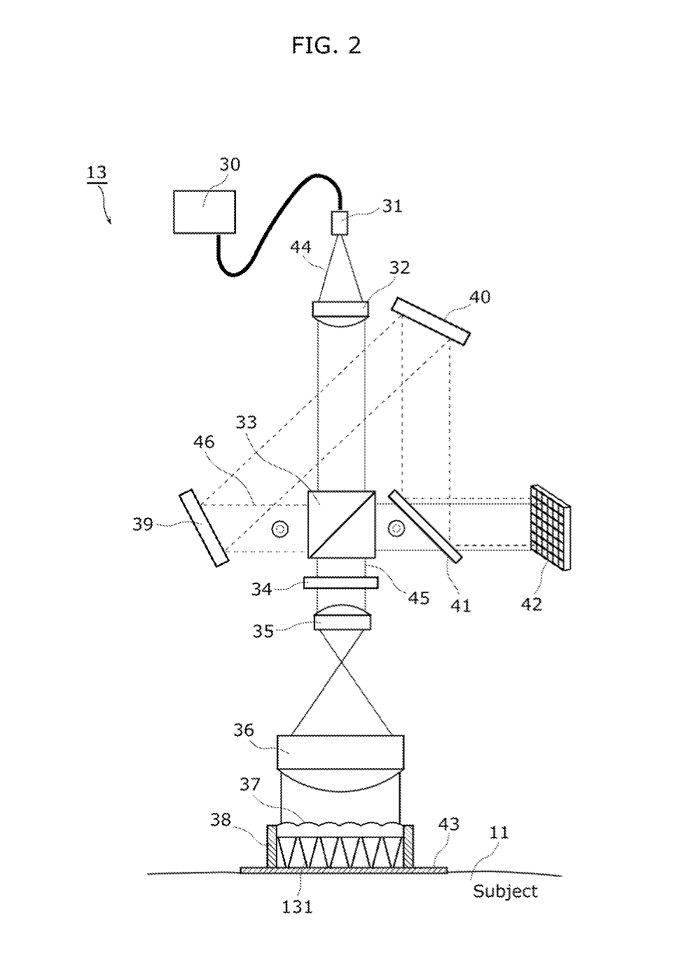

FIG. 2 shows the specific configuration of the optical probe 13 in Embodiment 1 of the present invention.

In FIG. 2, the optical probe 13 includes: a light source; an optical system which irradiates the surface of the subject with light emitted by the light source; and a light detector 42 which receives light reflected on the surface of the subject.

Specifically, the light source includes: a semiconductor laser 31 having an operation period in which an injected current and an outgoing waveform locally change in a linear fashion; and a current modulator 30 which modulates the current to be supplied to the semiconductor laser 31. The optical system includes: a collimating lens 32 which collimates laser light emitted by the semiconductor laser 31; a polarizing beam splitter 33 which transmits a P polarization component and reflects an S polarization component; a quarter wavelength plate 34 which coverts, into circularly polarized light, linearly polarized light transmitted by the polarizing beam splitter 33; lenses 35 and 36 for increasing a diameter of a luminous flux; a lens array 37; a spacer 38 for keeping a constant distance between the subject 11 and the lens array 37; mirrors 39 and 40 which reflect light reflected by the polarizing beam splitter 33; and a half mirror 41. The light detector 42 corresponds to a light-receiving element in an implementation of the present invention and receives the light reflected from a surface of the subject, with a plurality of photoelectric converters arranged in two dimensions. Furthermore, the optical probe 13 includes a reflector 43 which is disposed on a surface of the subject 11 to convey displacement of the surface of the subject 11.

Here, the reflector 43 corresponds to a reflector in an implementation of the present invention and is disposed on a surface of the subject 11 in close contact with the surface of the subject 11 to reflect light emitted onto the subject 11 according to vibration of the surface of the subject 11 caused by the ultrasound waves reflected off the internal tissue of the subject 11. With this structure, the light emitted from the light source is directed to the reflector, which means that the subject 11 is not directly irradiated with the light emitted from the light source and therefore can be kept safe. In addition, since the light emitted from the light source is reflected by the reflector, it is possible to reduce attenuation of the emitted light and thereby obtain a sufficient amount of light.

The semiconductor laser 31 is configured to superimpose a sawtooth current onto an injected current using the current modulator 30 and thereby emit laser light 44 generated by modulating the emitted light to have a sawtooth optical frequency. It is to be noted that the modulation frequency of wavelength of light will be hereinafter referred to as optical frequency so that it will be distinguished from modulation of intensity (amplitude). Here, the laser light 44 emitted by the semiconductor laser 31 has the P polarization component and the S polarization component to the polarizing beam splitter 33 so that the laser light 44 is partially transmitted and partially reflected by the polarizing beam splitter 33, thereby being separated into transmitted light 45a and reference light 46.

The spacer 38 is configured to have a length substantially equal to a focal length of the lens array 37 so that the light emitted from the lens array 37 is collected on the reflector 43 and the receiving spots 131 are thus formed.

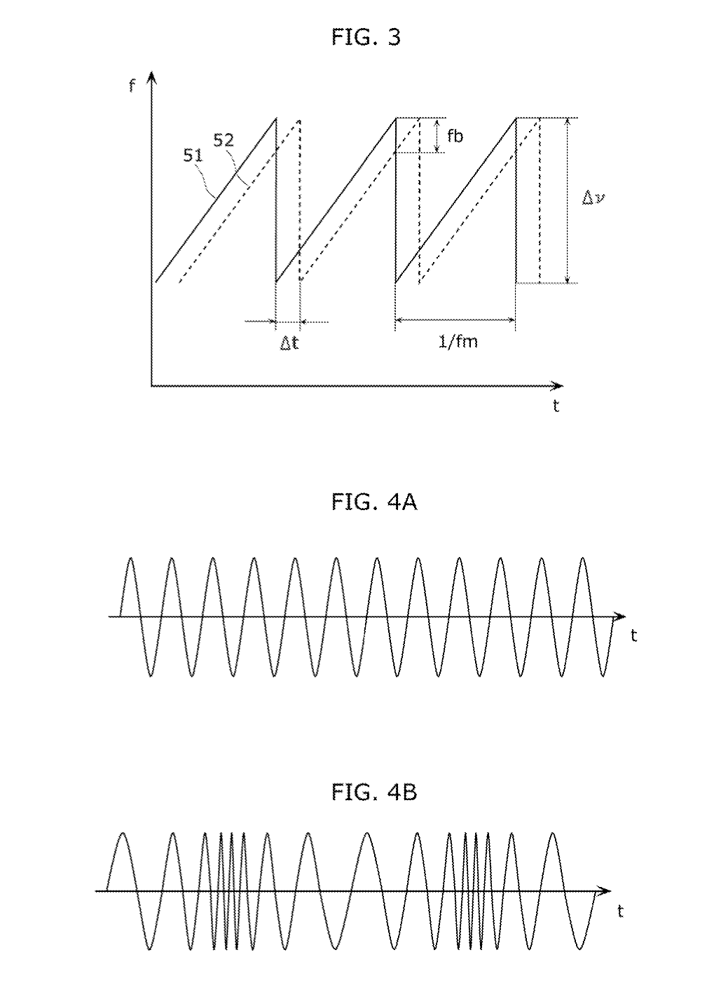

FIG. 3 illustrates signal waveforms of the reference light and the detection light which are detected by the light detector 42 in Embodiment 1 of the present invention. Here, in FIG. 3, the vertical axis represents the optical frequency and the horizontal axis represents time.

As shown in FIG. 3, a signal waveform 51 of the reference light and a signal waveform 52 of the detection light are waveforms shifted by time Δt. This is because there is an optical path difference between the reference light and the detection light; that is, they are different in path which they travel after the separation in the polarizing beam splitter 33 until they reach the light detector 42. Here, assuming that the distance between the polarizing beam splitter 33 and the surface of the subject 11 is denoted by L/2, and the light speed is denoted by c, the optical path difference will be L, which means that the signal waveforms are different by Δt=L/c. At this time, there is a slight difference in the optical frequency between the reference light and the detection light which are received by the light detector 42, and the light detector 42 therefore detects a beat signal of this difference frequency (hereinafter referred to as beat frequency) fb=Δv·fm·Δt. For example, assuming that the repetition frequency of sawtooth waves of the laser light 44 is fm=10 MHz, the optical frequency fluctuation range is Δv=15 GHz, and the optical difference between the reference light and the detection light is L=40 mm, the beat frequency fb will be 20 MHz.

In the case where the surface of the subject 11 vibrates because of propagation of the ultrasound echoes 28, the detection light reflected on the surface of the subject 11 will have a slightly shifted optical frequency due to the Doppler shift, with the result that the beat signal detected by the light detector 42 is likewise influenced by the Doppler shift. The signal detected by the light detector 42 will therefore be an FM signal with the beat signal as the center frequency, and by demodulating this FM signal, it is possible to detect the vibration caused by the ultrasound echoes 28 reflected inside the subject 11.

Here, for example, assuming that the variable amplitude of the surface of the subject 11 under the influence of the ultrasound echoes 28 is 0.5 nm and the frequency of the ultrasound echoes 28 is 5 MHz, the fluctuation velocity v of the surface of the subject 11 will be up to 0.0157 m/s. At this time, assuming that the wavelength in the light source is λ=683 nm, the frequency fb of the Doppler shift is determined by fb=4πv/λ=289 kHz. Thus, when the beat frequency is 20 MHz, the frequency of the signal detected by the light detector 42 is between 19.71 MHz and 20.29 MHz, which results from frequency modulation (FM). This is described with reference to the drawings.

FIGS. 4A and 4B each illustrate an output waveform of the light detector 42 in Embodiment 1 of the present invention. FIG. 4A shows the case with no Doppler shift while FIG. 4B shows the case with the Doppler shift.

In the case where there is no vibration in the surface of the subject 11 with no ultrasound waves transmitted to the inside of the subject 11, carrier waves, as shown in FIG. 4A, of a beat frequency depending on an optical path difference L between the reference light and the detection light are detected. On the other hand, in the case where the surface of the subject 11 vibrates with the ultrasound waves transmitted to the inside of the subject 11, carrier waves are frequency-modulated as shown in FIG. 4B, which means that, by demodulating this signal, it is possible to detect the vibration (hereinafter referred to as an echo signal) of the surface of the subject caused by the ultrasound echoes 28.

Next, the operation of the ultrasound examination device 10 configured as above is specifically described with reference to FIGS. 1A and 1B.

With reference to FIG. 1A, the control unit 19 first controls the transmitting unit 18 so that the transmitting unit 18 generates a drive signal with predetermined timing. The transmitting unit 18 provides, to each of the plurality of transducers of the ultrasound probe 12, drive pulses processed with a delay for focusing and deflecting the ultrasound waves which the ultrasound probe 12 transmits.

According to the drive pulses transmitted from the transmitting unit 18, the ultrasound probe 12 forms a predetermined wavefront by transmitting the ultrasound waves 26 from each of the transducers. These ultrasound waves 26 travel in a predetermined direction according to this wavefront. The ultrasound waves 26 transmitted from the ultrasound probe 12 are then reflected on an interface of tissue having different acoustic impedances inside the subject 11 and propagate, in form of the ultrasound echoes 28, to the surface of the subject 11, thereby conveying, to the reflector 43, the vibration caused by the ultrasound waves. The ultrasound echoes 28 conveyed to the reflector 43 have their vibration displacement detected by the transducers 121, which are a plurality of receiving points of the optical probe 13, and are received as an echo signal.

Here, the echo signal detecting operation at this time is described with reference to the drawings. First, as shown in FIGS. 2 and 3, the current modulator 30 modulates the injected current so that the semiconductor laser 31 emits the laser light 44 with the modulated optical frequency. This laser light 44 is converted into parallel light by the collimating lens 32 and enters the polarizing beam splitter 33 whereby the laser light 44 is separated into the detection light (the transmitted light) 45 having the P polarization component and the reference light (the reflected light) 46 having the S polarization component. The detection light 45, which serves as light for examination, is converted into the circularly polarized light by the quarter wavelength plate 34 and has its luminous flux increased by the lenses 35 and 36. The circularly polarized detection light 45 with an increased luminous flux passes through the lens array 37 to form a plurality of spots on the reflector 43. Meanwhile, the reference light 46 is led to the half mirror 41 by the mirrors 39 and 40. It is to be noted that, because of the separation into the detection light (the transmitted light) 45 and the reference light (the reflected light) 46 in this polarizing beam splitter 33, the reference light and the detection light can be separated at a position near the subject 11, which produces an effect of reducing vibration (body motion) noise.

When the vibration caused by the ultrasound echoes 28 is conveyed to the surface of the subject 11 at this time, the detection light 45 reflected by the reflector 43 will have a slightly shifted optical frequency due to the Doppler shift. The detection light 45 having the slightly shifted optical frequency then passes through the lenses 36 and 35 again to enter the quarter wavelength plate 34 whereby the detection light 45 is converted into linearly polarized light having the S polarization component, and then enters the polarizing beam splitter 33 whereby the detection light 45 is reflected to enter the half mirror 41. As a result, the detection light 45 transmitted by the half mirror 41 and the reference light 46 reflected by the half mirror 41 are multiplexed, and such multiplexed light enters the light detector 42. The light detector 42 receives this multiplexed light to detect a signal such as the beat signal.

The signal detected as above is a frequency-modulated (FM) signal which has, as the center frequency, the beat frequency depending on the optical path difference between the reference light and the detection light, and by demodulating the FM signal in the receiving unit 14, it is possible to obtain a detection signal of the ultrasound echoes 28.

With reference back to FIG. 1A, the description continues. A plurality of echo signals demodulated by the receiving unit 14 and further amplified are converted into digital signals and stored in the storing unit 15a. Here, on the basis of the echo signals stored in the storing unit 15a, the arithmetic processing unit 15b performs the phasing addition, that is, the beam forming processing for the region along the path for transmission (hereinafter referred to as a sound ray) of the ultrasound waves 26, and obtained data on the sound ray are stored in the image memory 15c.

The above operation is performed while scanning the inside of the subject with the sound ray of the ultrasound waves 26 transmitted from the ultrasound probe 12, and information on the entire region to be examined is computed and stored in the image memory 15c. The three-dimensional data stored in the image memory 15c are rendered as a three-dimensional image by the image processing unit 16, and the resultant image is displayed by the image display unit 17.

Next, the resolution of the ultrasound examination device 10 configured as above is described. Here, the azimuth resolution can be regarded as the shortest possible distance at which a phase difference is distinguishable between echo signals coming from two reflection points located in the azimuth direction. A condition of this azimuth resolution is described with reference to the drawings.

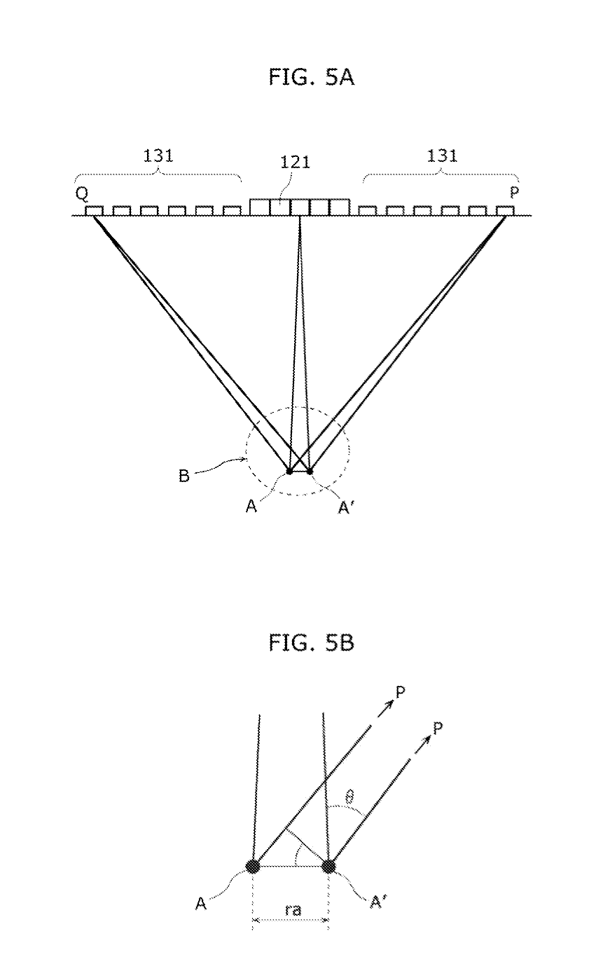

FIGS. 5A and 5B each illustrate the resolution of the ultrasound examination device 10 in Embodiment 1 of the present invention. FIG. 5A illustrates the azimuth resolution, and FIG. 5B shows a closeup of a B part of FIG. 5A. In FIG. 5B, ra denotes a A-A′ distance, and θ denotes an elevation angle of each direction from points A and A′ to both ends P and Q of the receiving spots 131. Here, when the distance from the reflection points A and A′ to the transducers 121 is long enough compared to ra, the elevation angles θ for A and A′ can be assumed to be the same.

Here, assuming that point P and point Q are at symmetrical positions, approximate values are obtained as follows: AP−A′P=A′Q−AQ=ra·sin θ. Furthermore, assuming that a difference in phase of the echo signals can be detected using a phase difference n, the resolution is obtained by ra=λ/(2·sin θ). Here, λ denotes a wavelength of the ultrasound waves.

This shows that the azimuth resolution is proportional to the wavelength λ and inversely proportional to sine. Although a decrease in the wavelength λ, that is, an increase in the frequency, will improve the azimuth resolution, there is a problem of an increase in the attenuation inside the subject. Thus, in this embodiment, the openings of the receiving spots 131 of the optical probe are expanded with the use of light detection as described above so that θ can be larger. In the above-described manner, this embodiment produces an effect of providing good azimuth resolution as compared to a conventional structure.

Furthermore, since the receiving openings are expanded to improve the resolution, the ultrasound beams to be transmitted can be relatively wide, resulting in a wider scanning pitch. It is therefore possible to reduce the number of times to transmit and receive the ultrasound waves. This allows a decrease in the length of shooting time.

As described above, the ultrasound examination device 10 according to Embodiment 1 produces an effect of significantly improving the resolution by providing the optical probe 13 for receiving the ultrasound waves with the openings larger than the openings of the ultrasound probe 12 for transmitting the ultrasound waves. In addition, this allows an increase in the width of the ultrasound beams to be transmitted and also an increase in the width of the scanning pitch, which produces an effect of decreasing the length of shooting time.

Furthermore, with the structure of detecting the ultrasound echoes with use of light, it is easy to provide the structure in which a large number of receiving spots are arranged in two dimensions.

In this embodiment, the heterodyne interference optical system is applied to detect the vibration of the surface of the subject, and the laser light emitted from the light source is modulated so as to have a sawtooth optical frequency so that the beat signal depending on the optical path difference between the reference light and the detection light is detected. However, it may also be possible that, after the splitting into the reference light and the detection light, the beat signal is generated by shifting the optical frequency of at least one of the reference light and the detection light using a general structure such as an acousto-optic modulator (AOM).

Furthermore, although the ultrasound probe 12 performs only transmission of the ultrasound waves in this embodiment, the present invention is not limited to this embodiment. The ultrasound probe 12 may be configured to perform calculations not only according to the drive pulses transmitted from the transmitting unit 18, but also using a signal received by the ultrasound probe 12, to improve the S/N ratio of the ultrasound waves 26 to be transmitted from each transducer.

Furthermore, the above describes, as an example, a structure in which the ultrasound vibration of the surface of the subject 11 is detected by a heterodyne interference optical system which serves as the optical probe 13. Although the above describes the example in which the heterodyne interference optical system is disposed in a probe for examination, the present invention is not limited to this example. Specifically, for example, this optical system is not necessarily disposed in the probe for examination and may be disposed on a main device side and configured to transmit and receive the detection light using an optical fiber or the like. This case is described below.

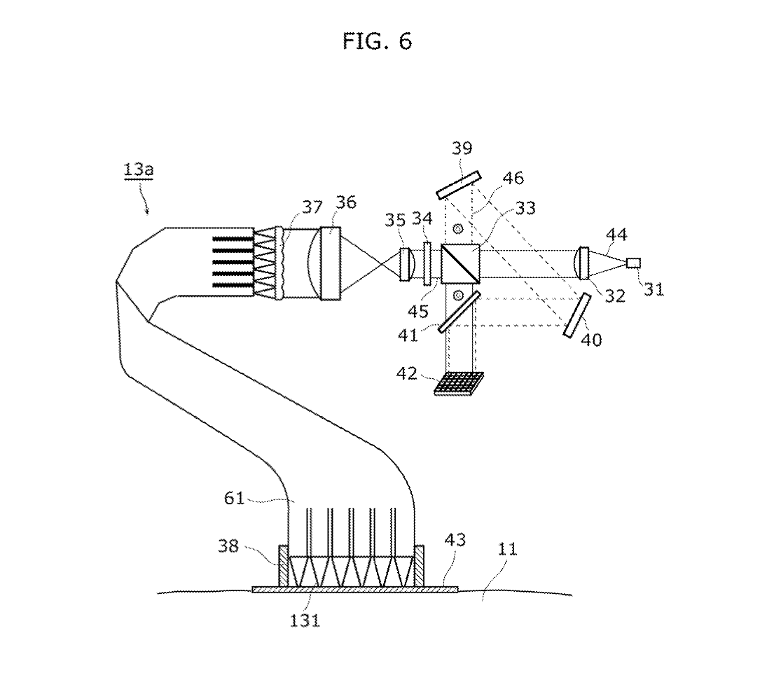

FIG. 6 shows a variation of the specific configuration of the optical probe in Embodiment 1 of the present invention. Specifically, an optical probe 13a shown in FIG. 6, is a variation of the optical probe 13 including the heterodyne interference optical system shown in FIG. 2. The only difference in FIG. 6 from FIG. 2 is a structure of an optical fiber bundle 61. Although not shown, there is a gradient-index (GRIN) lens at an end of the optical fiber bundle 61 on the subject 11 side so that the light is collected on the reflector 43.

With this structure, the heterodyne interference optical system can be disposed not on the probe side but on the diagnostic device side, which produces an effect of reducing the size of the probe for examination. Furthermore, even when a plurality of optical probes 13 are provided as the probe included in the diagnostic device, the detection light from respective positions is collected to a single place by use of the optical fiber bundle 61, with the result that signals from a plurality of places can be detected by a single heterodyne interference optical system. This further produces an effect of reducing the size of the diagnostic device and the manufacturing cost of the diagnostic device.

Furthermore, the above describes an example in which the heterodyne interference optical system is used as an optical system for detecting a small displacement and vibration of the subject, the present invention is not limited to this example. For example, a configuration using the Fabry-Perot resonator structure or other interference optical system may be used. Alternatively, it may also be possible that the reflector 43 is configured to change the optical properties due to a small distortion generated by propagation of the ultrasound waves, and this change in the optical properties is detected with use of light. In either case, the detection of ultrasound waves with use of light allows a decrease in the electrical connections, which produces an effect of easily increasing the number of receiving spots 131.

Furthermore, the optical probe 13 may be configured to detect ultrasound vibration of the internal tissue of the subject 11, for example. This structure example is described below.

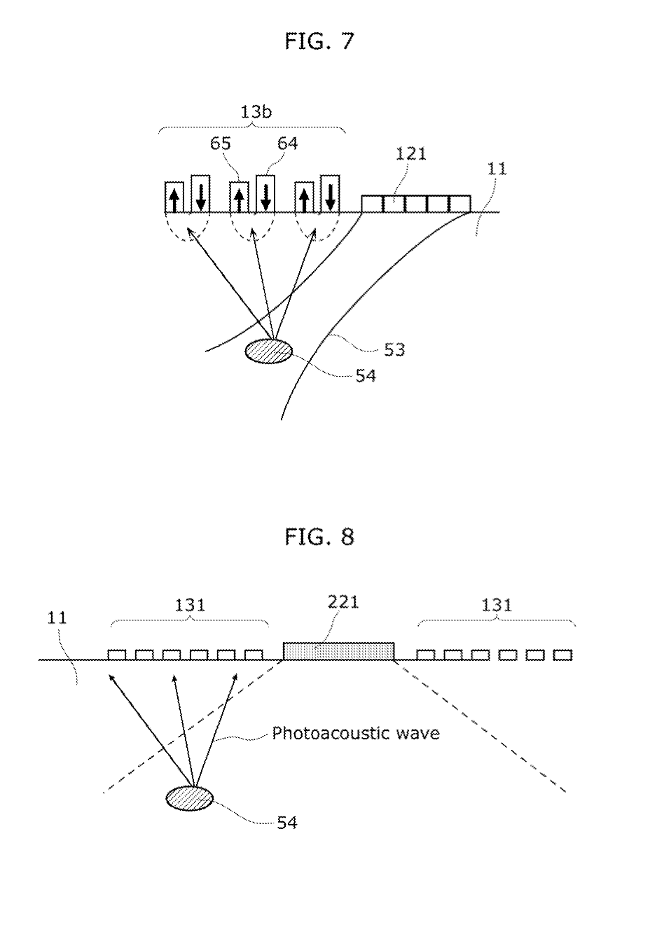

FIG. 7 shows another specific configuration of the probe for examination in the ultrasound examination device in Embodiment 1 of the present invention.

In FIG. 7, the optical probe 13b includes: a transmitting fiber 64 configured to transmit broadband light to the inside of the subject 11; and a receiving fiber 65 which detects the broadband light transmitted through the subject. Although not shown, the transmitting fiber 64 is connected to a broadband light source for emitting (transmitting) light of a plurality of wavelengths, and the receiving fiber 65 is connected to a spectrometer and a light-receiving element so that absorption of light can be detected. The light source is not limited to the broadband light source as long as it emits light of a plurality of wavelengths.

In this structure, an arrival of the ultrasound echoes 28 at the vicinity of the surface of the subject causes a change in the optical properties of tissue whose subcutaneous tissue undergoes scattering and absorption. Since an amount of the change reflects the local optical properties, the ultrasound waves can be detected from a change in the absorption of light obtained from the detection light.

In the drawing, an amount (a length of a vector) of light which is received through the receiving fiber 65 is shorter than an amount (a length of a vector) of light which is transmitted through the transmitting fiber 64. This is because the light transmitted to the inside of the subject 11 passes through the subject 11 before reaching the receiving fiber 65, with the result that the light is attenuated inside the subject 11.

With this structure, the optical probe 13 can be composed of the fiber only, which produces an effect of significantly reducing the size of the probe for examination included in the ultrasound examination device.

As the structure of detecting the ultrasound vibration of the internal tissue of the subject 11, another structure example is conceivable which is different from the probe for examination in the ultrasound examination device shown in FIG. 7. For example, a conceivable structure is such that, instead of the transducers 121 of the ultrasound probe 12 for transmitting the ultrasound waves, a light source 221 for transmitting acoustic waves is provided in the probe for examination in the ultrasound examination device. This structure example is described below.

FIG. 8 shows another configuration of the probe for examination in the ultrasound examination device in Embodiment 1 of the present invention. FIG. 8 shows a schematic configuration of an examination device which uses the photo acoustic tomography (PAT) technique called photoacoustic imaging. This photoacoustic imaging is implemented as one of the optical imaging devices which emit laser light or the like to a living body (the subject 11) and provide an image of information about an inside of the living body obtained based on the incident light.

In this device, pulsed light generated from the light source 221 is emitted to a living body (the subject 11), and acoustic waves generated from body tissue which absorbed energy of the pulsed light propagated and diffused inside the living body are detected by the receiving spots 131. Specifically, using a difference in the light energy absorption rate between a subject site, such as a tumor, and the other tissue, the acoustic waves generated from the body tissue are detected by receiving elastic waves which are generated upon momentary dilation of the subject site when absorbing the light energy emitted thereto. By analyzing this detected signal, it is possible to obtain distribution of optical properties, especially, light energy absorption and density distribution, of the living body.

Also in the device configured as above, the acoustic waves generated from the body tissue can be detected using the optical probe with large openings, which produces an effect of detecting the body tissue, such as a tumor, with high resolution.

Next, yet another specific configuration of the optical probe 13 in Embodiment 1 of the present invention is described.

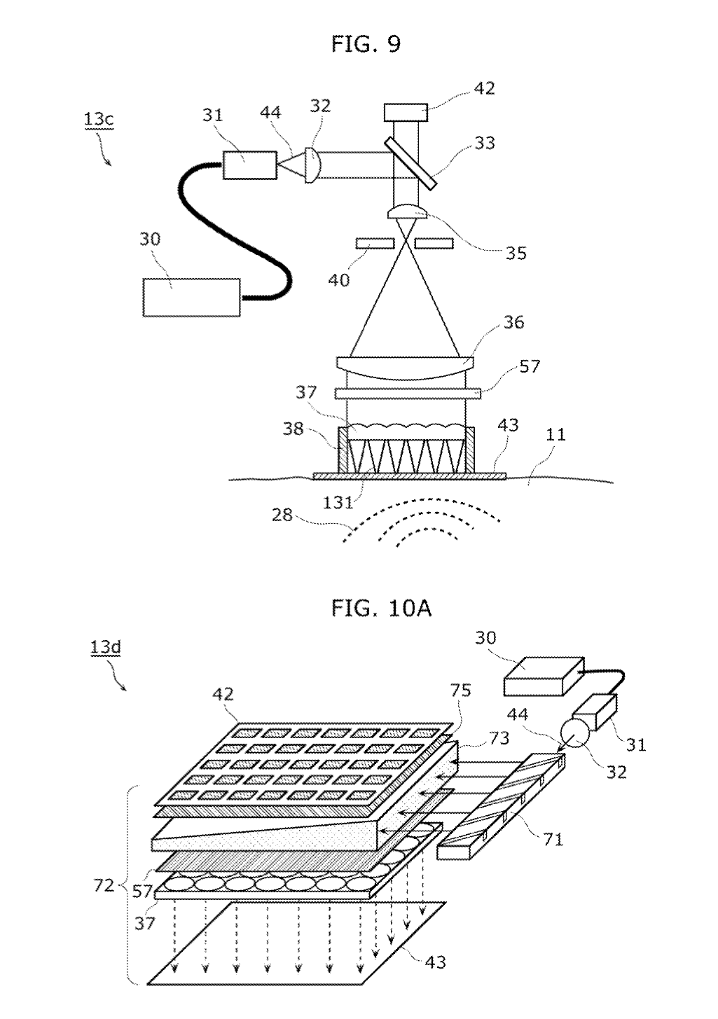

FIG. 9 shows a variation of the specific configuration of the optical probe in Embodiment 1 of the present invention. Constituents which are the same or like as those in FIG. 2 are denoted with the same signs and will not be described in detail.

In FIG. 9, an optical probe 13c includes: the semiconductor laser 31 having an operation period in which an injected current and an outgoing waveform locally change in a linear fashion; the current modulator 30 which modulates the current to be supplied to the semiconductor laser 31; the collimating lens 32 which collimates the laser light 44 emitted by the semiconductor laser 31; the polarizing beam splitter 33 which transmits the P polarization component and reflects the S polarization component; a beam expander composed of the lenses 35 and 36; a polarization reflector 57 composed of, for example, a wire grid polarizer, which transmits polarized light in the transmission axis direction and reflects a polarization component orthogonal to the polarized light; the lens array 37 which collects, on the subject 11, the light transmitted by the polarization reflector 57, to form the plurality of receiving spots; a reflector 43 disposed on the subject 11; and the light detector 42 having a plurality of light-receiving regions corresponding one-to-one with the receiving spots on the reflector 43.

The semiconductor laser 31 is configured to superimpose a sawtooth current onto an injected current using the current modulator 30 and thereby emit the laser light 44 with the modulated optical frequency.

The polarization reflector 57 is configured such that the transmission axis is inclined at approximately 45 degrees with respect to the S polarized light in the polarizing beam splitter 33 so that the light incident on the polarization reflector 57 is partially reflected and partially transmitted.

The lens array 37 is configured to form the plurality of receiving spots so that these receiving spots may either be arranged in a single line in a predetermined direction or be arranged in a two-dimensional matrix.

When the detection light reflected on the receiving spot on the reflector 43 re-enters the lens array 37, the detection light which enters a lens different from the lens through which the light has passed does not enter a corresponding one of the light-receiving regions on the light detector 42. Such light becomes stray light and is to be intercepted by an aperture 40.

A structure of a conventional heterodyne interference optical system is a structure of separating the reference light inside a measurement device and therefore has a problem of S/N ratio deterioration which is due to addition of noise generated by vibration between the measurement device and an object to be measured to information on displacement of the object itself. In contrast, a structure of the optical probe 13c shown in FIG. 9 which includes the heterodyne interference optical system is a structure of detecting relative displacement, vibration, and the like, with respect to the polarization reflector 57 composed of a wire grid polarizer, for a example. It is therefore possible to reduce the noise which is generated by vibration in a surrounding environment.

The following describes a specific configuration which is different from the optical probe shown in FIG. 2 or FIG. 9 which includes the heterodyne interference optical system.

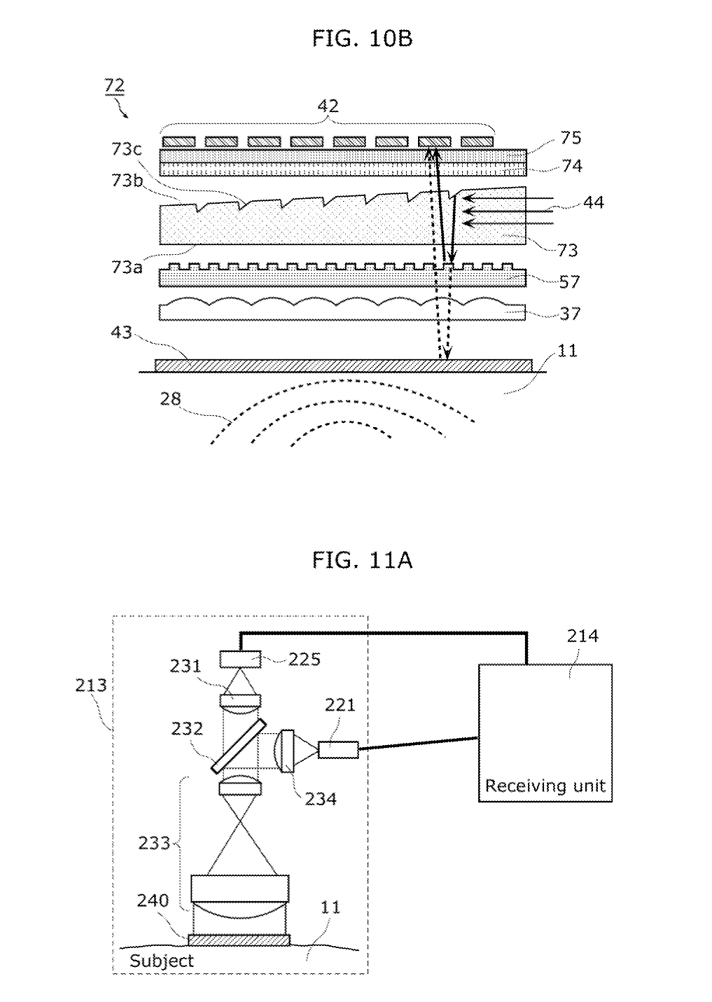

FIGS. 10A and 10B each show another specific configuration of the optical probe in Embodiment 1 of the present invention. An optical probe 13d shown in FIGS. 10A and 10B includes a thin heterodyne interference optical system. Here, FIG. 10A is a perspective view, and FIG. 10B is a cross-sectional view of a main part. Constituents which are the same or like as those in FIG. 2 are denoted with the same signs and will not be described in detail.

In FIG. 10A, the optical probe 13d includes, as an optical system in an implementation of the present invention, the semiconductor laser 31, the current modulator 30, the collimating lens 32, a light guide rod 71 for converting the laser light 44 into linear parallel light, and a planar detection unit 72.

The light guide rod 71 has a structure which includes a plurality of deflection grooves each having an inclined surface inclined at approximately 45 degrees with respect to a side surface from which light is emitted, and totally reflects the light incident on the light guide rod 71 to deflect the light at approximately 90 degrees.

The planer detection unit 72 has a structure shown in FIG. 10B, for example. Specifically, the planer detection unit 72 includes: a light guide plate 73 on whose side surface the laser light 44 emitted from the light guide rod 71 is incident and from whose main surface 73a the laser light 44 is emitted; the polarization reflector 57 disposed adjacent to the main surface 73a of the light guide plate 73; the lens array 37; a polarizer 74 disposed opposite to the polarization reflector 57 across the light guide plate 73; a viewing angle control sheet 75 which transmits only the substantially vertically incident light out of the light transmitted by the polarizer 74; and the light detector 42 having the light-receiving regions corresponding to the light-receiving spots on the subject 11.

In an opposite surface 73b opposite to the main surface 73a in the light guide plate 73, a plurality of deflection surfaces 73c are formed each of which is an inclined surface inclined at approximately 45 degrees with respect to the main surface 73a. The light guide plate 73 is configured to totally reflect the light incident in substantially parallel on the main surface 73a, to deflect the light in a direction which is perpendicular to the main surface 73a, and then emit the light substantially vertically from the main surface 73a.

The polarizer 74 is configured to have a transmission axis which forms an angle of approximately 45 degrees with the transmission axis of the polarization reflector 57.

The viewing angle control sheet 75 is disposed with the aim of preventing stray light from entering each of the light-receiving regions of the light detector 42. Here, the stray light is light which comes from a region other than a corresponding one of the receiving spots.

Subsequently, the operation of the optical probe 13d configured as above is described.

First, in the optical probe 13d, the laser light 44 with the modulated optical frequency is collimated by the collimating lens 32, is converted to linear parallel light by the light guide rod 71, and enters the planar detection unit 72. The laser light 44 incident on the light guide plate 73 of the planar detection unit 72 is deflected by the deflection surface 73c, is emitted substantially vertically from the main surface 73a of the light guide plate 73, and is then separated into the reference light and the detection light by the polarization reflector 57.

Next, the detection light reflected on the plurality of receiving spots which are formed on the surface of the subject 11 by the lens array 37, and the reference light reflected on the polarization reflector 57 pass through the light guide plate 73 and enter the polarizer 74. Here, the polarized light of the reference light and the polarized light of the detection light are orthogonal to each other; both the polarized light is at approximately 45 degrees with respect to the transmission axis of the polarizer 74. Accordingly, the light of the same polarization component passes through the polarizer 74, and only the substantially vertically transmitted light passes through the viewing angle control sheet 75 and interferes with each other on the light detector 42, thereafter being detected in each of the light-receiving regions of the light detector 42.

As above, by demodulating the detected FM signal, the as vibration at each of the receiving spots can be detected. It is therefore possible to implement a configuration of the optical probe which is small and thin in size and provides high resolution. This produces an effect of providing an ultrasound examination device which provides high resolution.

### Embodiment 2

While Embodiment 1 describes an example in which the optical probe is composed of the heterodyne optical system, Embodiment 2 describes an example in which the optical probe is composed of the Fabry-Perot resonator structure.

FIGS. 11A and 11B each show configurations of an optical probe and a receiving unit in Embodiment 2 of the present invention. FIG. 11A shows a configuration in which the light source 221 and the light-receiving element are disposed inside an optical probe 213, and FIG. 11B shows a configuration in which the light source 221 and the light-receiving element, i.e., a light detector 225, are disposed inside the device main body, i.e., a receiving unit 214, of the ultrasound examination device (the ultrasound diagnostic device). Here, FIG. 11B shows a specific configuration example of the configuration shown in FIG. 11A. This means that the configuration and operation in FIG. 11A are the same as those in FIG. 11B except that illuminating light and detection light are conveyed with use of an optical fiber, and the following descriptions are therefore made with reference to FIG. 11B.

As shown in FIG. 11B, the optical probe 213 and the receiving unit 214 are connected to each other with use of an optical fiber 220 and an image fiber 230 in which several tens of thousands of optical fibers are placed so that an image can be conveyed.

The receiving unit 214 includes: the light source 221 that is a laser light source capable of a wavelength locking operation at a given wavelength around 1,000 nm in a narrow wavelength band (of 50 pm, for example); and a condensing lens 223 which collects laser light 222 emitted from the light source 221, and the receiving unit 214 sends the laser light 222 through the optical fiber 220. Furthermore, the receiving unit 214 includes: a magnifying lens 224 which magnifies the image sent through the image fiber 230; and the light detector 225 having a two-dimensional array composed of a charge-coupled device (CCD), a metal oxide semiconductor (MOS) sensor, or a plurality of photodiodes (PD), for receiving light of the magnified image.

The optical probe 213 includes: a collimating lens 231 which converts, into parallel light, the laser light 222 sent through the optical fiber 220; a half mirror 232 by which the incoming laser light 222 and the outgoing laser light 222 travel in different routes; a beam expander 233; an ultrasound detection element 240 disposed on a surface of the subject 11; and an image-forming lens 234 which leads, into the image fiber 230, the laser light 222 reflected by the ultrasound detection element 240.

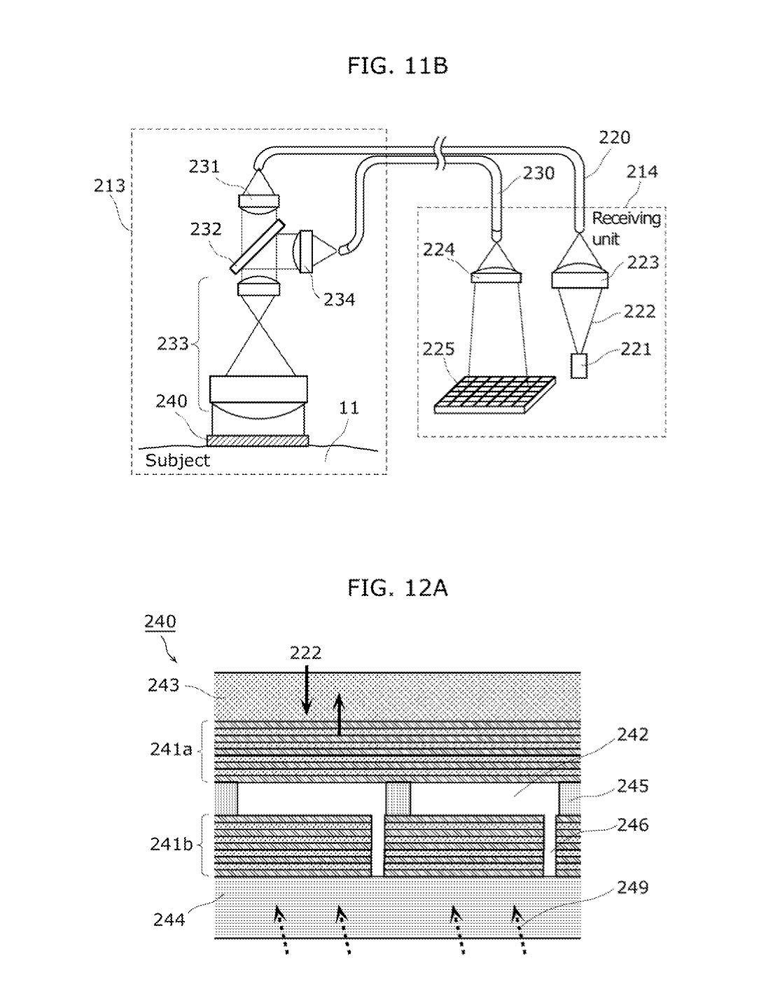

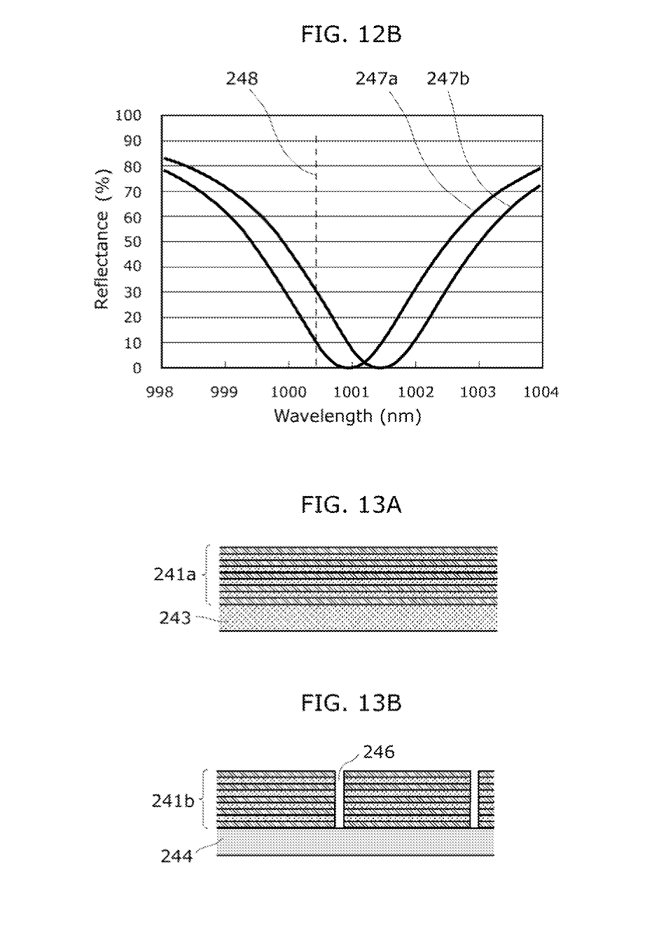

The ultrasound detection element 240 is composed of the Fabry-Perot resonator in which a resonator is formed of a multilayer mirror as shown in FIG. 12A, for example. Here, FIG. 12A schematically shows a configuration of the ultrasound detection element 240, and FIG. 12B shows spectroscopic properties of the ultrasound detection element 240 shown in FIG. 12A. In FIG. 12B, the horizontal axis represents the wavelength (nm) and the vertical axis represents the reflectance (%).

The ultrasound detection element 240 corresponds to a reflector in an implementation of the present invention and includes: a sound matching material 244 that has a plurality of regions, is formed in close contact with the subject 11, and converges, for each of a plurality of detection regions, the ultrasound waves representing vibration of the surface of the subject 11; and a first reflector element that is formed on a surface of the sound matching material 244 which is different from the surface in close contact with the subject 11, and reflects light emitted to the subject 11. Specifically, the ultrasound detection element 240 includes, as shown in FIG. 12A, a multilayer mirror 241a and a multilayer mirror 241b, an air layer 242, a transmissive substrate 243, and the sound matching material 244, which are stacked one after another. Here, the sound matching material 244 corresponds to an ultrasound converging unit in an implementation of the present invention and converges, for each of the plurality of detection regions, the ultrasound waves representing the vibration of the surface of the subject 11 and conveys the converged ultrasound waves to the first reflector element. The multilayer mirror 241a and the multilayer mirror 241b, the air layer 242, and the transmissive substrate 243 correspond to the first reflector element in an implementation of the present invention. The first reflector element amplifies the conveyed ultrasound waves and reflects the light emitted to the subject 11, with reflectance which varies depending on the amplified ultrasound waves.

The multilayer mirror 241a and the multilayer mirror 241b correspond to the first multilayer and the second multilayer, respectively, in an implementation of the present invention and are each formed by alternately stacking layers which have different refractive indices. Specifically, the multilayer mirror 241a and the multilayer mirror 241b are each formed by alternately stacking a low-refractive layer made of a low-refractive material (such as silicon dioxide (SiO₂)) and a high-refractive layer made of a high-refractive material (such as titanium dioxide (TiO₂)). Here, the multilayer mirror 241b includes a slit 246 so that deformation of the sound matching material 244 can be easily transferred.

A spacer 245 is formed (located) between the multilayer mirror 241a and the multilayer mirror 241b and fixes an end of the multilayer mirror 241b divided by the slit 246. The spacer 245 forms a space between the multilayer mirror 241a and the multilayer mirror 241b. Specifically, with the spacer 245, the air layer 242 between the multilayer mirror 241a and the multilayer mirror 241b has a constant thickness.

The slit 246 is formed so as to divide the multilayer mirror 241b into a plurality of regions (hereinafter referred to as detection regions), and the spacer 245 is disposed at an end of this detection region. With this structure, the detection region is in a cantilever state with the spacer 245 as a fulcrum, with the result that displacement is more likely to take place in an area opposite to the area in which the spacer 245 is located. Here, the cantilever state indicates a state in which all load is supported at an end while the other end is completely free.

Furthermore, the multilayer mirror 241a and the multilayer mirror 241b are configured such that, of each layer therein, an optical layer thickness determined by multiplying a physical layer thickness by a refractive index is substantially equal to a quarter of a set wavelength (e.g. 1,000 nm). Meanwhile, the air layer 242 is configured so as to have the optical layer thickness substantially equal to a half of the set wavelength in a default state and is configured to vary in thickness upon deformation of the sound matching material 244.

With this structure, the ultrasound detection element 240 propagates the ultrasound waves converged by the sound matching material 244, to an area near the other unfixed end of the divided multilayer mirror 241b so that the multilayer mirror 241b is deformed, and the propagated ultrasound waves are amplified.

In other words, the ultrasound detection element 240 is formed by alternately stacking layers which have different refractive indices, and includes the multilayer mirror 241a and the multilayer mirror 241b which have substantially identical reflection properties, and a substrate which the illuminating light emitted from the light source enters, and the multilayer mirror 241a is disposed opposite to the multilayer mirror 241b so as to form a resonator structure. The ultrasound detection element 240 propagates the ultrasound waves converged by the sound matching material 244, thereby varying a resonator length of the resonator structure to vary an amount of reflected light originated from the light emitted to the subject 11.

The ultrasound detection element 240 configured as above have spectroscopic properties as shown in FIG. 12B; the reflectance is significantly low with respect to a particular wavelength. FIG. 12B shows calculation results of the reflectance with the multilayer mirror 241a of four SiO₂ layers and the multilayer mirror 241b of five TiO₂ layers. A spectroscopic property 247a represents a property obtained when the thickness of the air layer 242 is 500.0 m, and a spectroscopic property 247b represents a property obtained when the thickness of the air layer 242 is 500.5 nm. For example, it can be seen that, in the case where the ultrasound detection element 240 is irradiated with light having a wavelength indicated by a broken line 248, a 0.5 nm change in the thickness of the air layer 242 from 500 nm causes a change in the reflectance from 10% to 30%.

It is to be noted that the ultrasound detection element 240 can increase a change in the reflectance relative to an amount of a change in the thickness of the air layer 242 by increasing the number of layers of each of the multilayer mirror 241a and the multilayer mirror 241b. With this structure, it is possible to further increase the sensitivity for detecting a small displacement.

Next, the operation of the ultrasound examination device 10 according to this embodiment configured as above is described.

As in the case of Embodiment 1, first, the ultrasound waves which are focused in a predetermined region are transmitted from the ultrasound probe 12 toward the subject 11. At this time, the control unit 19 controls the transmitting unit 18 so that the transmitting unit 18 generates a drive signal with predetermined timing. The transmitting unit 18 provides, to each of the plurality of transducers of the ultrasound probe 12, drive pulses processed with a delay for focusing and deflecting the ultrasound waves which the ultrasound probe 12 transmits.

The ultrasound waves transmitted from the transducers of the ultrasound probe 12 are reflected on an interface of tissue having different acoustic impedances inside the subject 11 and arrive, as the ultrasound echoes 28, at the surface of the subject 11, thereby transferring, to the optical probe 213, the vibration caused by the ultrasound echoes 28. The optical probe 213 converts the vibration of the ultrasound echoes into variation in the reflectance, reflects the light sent from the receiving unit 214, and sends the reflected light (an image) to the receiving unit 214, and the receiving unit 214 detects an echo signal from the variation in reflected light amount distribution of the reflected light sent from the optical probe 213.

The echo signal detecting operation at this time is described in detail with reference to FIGS. 11A to 12B. First, the wavelength of the light to be emitted from the light source 221 of the receiving unit 214 is adjusted before examination. At this time, while changing the wavelength of the light to be emitted, an amount of light reflected by the ultrasound detection element 240 is monitored. The wavelength of the light to be emitted is then selected and fixed so that the reflectance in the ultrasound detection element 240 is around 10%, for example.

Next, the laser light 222 is emitted from the light source 221 to detect an echo signal. At this time, emission of the laser light 222 is controlled by the control unit 19 so that the amount of light increases according to an elapsed time from transmission of the ultrasound waves from the ultrasound probe 12.

The laser light 222 emitted with its light emission adjusted is supplied to the optical probe 213 through the condensing lens 223 and the optical fiber 220 and passes through the collimating lens 231 and the beam expander 233, thereby illuminating the ultrasound detection element 240.

At this time, as shown in FIG. 12A, the transfer of ultrasound echoes 249 from the subject 11 to the sound matching material 244 causes the sound matching material 244 to deform due to the vibration caused by the transfer and at the same time, causes the multilayer mirror 241b to deform, which varies the thickness of the air layer 242. Here, assuming that the thickness of the air layer 242 has varied by around 0.5 nm, the reflectance changes from 10% to 30% as shown in FIG. 12B, which means that, at the moment of reception of the ultrasound echoes 249, the amount of reflected light in the site where the ultrasound echoes 249 were received becomes three times larger locally.

Since the signal amplitude of the ultrasound echoes 249 decreases as time passes after transmission of the ultrasound waves, this accompanies a decrease in the change of the reflectance of the ultrasound detection element 240. Thus, in this embodiment, the light source 221 is controlled so that the emission of the laser from the light source 221 increases as time passes. Specifically, the control unit 19 controls the timing of transmitting the ultrasound waves from the ultrasound probe 12, and according to an elapsed time from transmission of the ultrasound waves from the ultrasound probe 12, the control unit 19 controls the amount of light to be emitted from the light source. This produces an effect of detecting variation in the amount of reflected light even when the amplitude of the ultrasound echoes 249 decreases.

Furthermore, in the case where the amount of reflected light before detection of the ultrasound echoes 240 is too large because of the control on the light emission of the light source 221 that is a laser light source, the wavelength of the laser light 222 may be shifted in the direction in which the reflectance decreases, as time passes.

Alternatively, the wavelength of the laser light 222 may be fixed, from the beginning of examination, at the wavelength with which the reflectance of the ultrasound detection element 240 is minimum. Specifically, it is sufficient that the optical probe 13 adjusts the wavelength of the light to be emitted from the light source (the semiconductor laser 31 and the current modulator 30), so as to minimize the reflectance in the ultrasound detection element 240, and then detects, with use of light, the ultrasound waves reflected off the internal tissue of the subject 11. With this structure, for example, by adjusting the wavelength so that the reflectance becomes zero, it is possible to regard even small variation in the amplitude of the ultrasound echoes 249 as large variation, that is, it is possible to improve the rate of change in the reflectance, which produces an effect of improving the receiving sensitivity.

The spectroscopic properties of the ultrasound detection element 240 may vary due to a manufacturing error in layer thickness of each of the multilayer mirror 241a and the multilayer mirror 241b or due to a surrounding environment. However, in this embodiment, a change in the detection sensitivity due to variation in the spectroscopic properties can be reduced by measuring the spectroscopic properties in advance and appropriately setting the wavelength of the light source 221 that is a laser light source. In short, according to this embodiment, it is possible to provide the ultrasound detection element 240 which is easy to manufacture and has high sensitivity.

In FIG. 11B, upon receiving the ultrasound echoes 28 (the ultrasound echoes 249 in FIG. 12) by the ultrasound detection element 240, ripples (intensity distribution) of the ultrasound echoes appear on the ultrasound detection element 240. An image of these ripples is sent to the receiving unit 214 through the beam expander 233, the half mirror 232, the image-forming lens 234, and the image fiber 230, and is magnified by the magnifying lens 224 and then is received, as two-dimensional intensity distribution, by the light detector 225. A data amount of the signal detected by the light detector 225 is adjusted as necessary, and the signal is then converted into an electric signal and sent, as an echo signal, to the signal processing unit 15.

While the image fiber 230 is capable of transmitting an image of several thousands of pixels, the number of pixels of the light detector 225 may be smaller. The light detector 225 is preferably set to have the appropriate number of pixels in consideration of a load of downstream signal processing. For example, the number of pixels of the light detector 225 may be several hundreds to several thousands. Even with this structure, it is possible to detect the echo signals in a wide range and with a large number of spots as compared to the case of detecting the ultrasound echoes 28 by a piezoelectric element.

Furthermore, the arrangement of pixels of the light detector 225 is not limited to a matrix. An unevenly dispersed (sparse) arrangement or a configuration in which pixels are different in shape, size, or the like is also applicable.

Furthermore, a large amount of the detected echo signals in the wide range are stored in the storage unit 15a as in the case of Embodiment 1. The beam forming processing is then performed in the arithmetic processing unit 15b, and three-dimensional data are stored in the image memory 15c. At this time, by processing the large amount of echo signals in the wide range, the resolution of the beam forming processing improves, and three-dimensional data with a high S/N ratio can be obtained.

By performing the above operation while changing the transmission direction of the ultrasound waves, the inside of the subject 11 is three-dimensionally scanned at high speed, and information on the entire examined region is computed and stored in the image memory 15c. The stored information on the entire examined region, that is, the three-dimensional data, is rendered as a three-dimensional image by the image processing unit 16, and the resultant image is displayed on the image display unit 17. Through such operation, an ultrasound image in a wide range can be obtained at high speed and with high resolution.

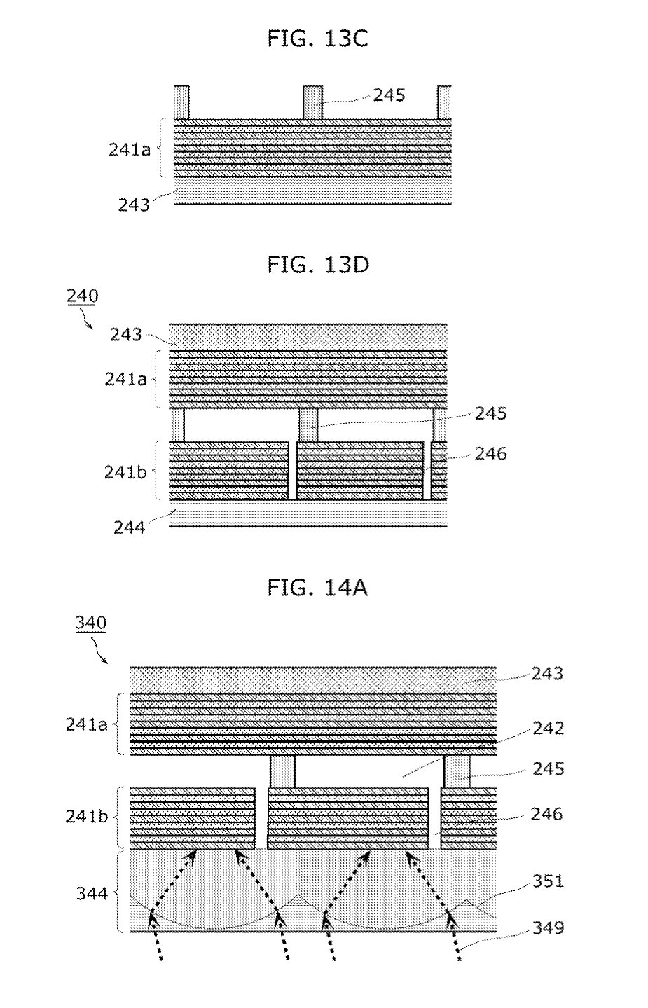

Next, with reference to FIGS. 13A to 13D, a method of manufacturing the ultrasound detection element 240 is described. FIGS. 13A to 13D illustrate the method of manufacturing the ultrasound detection element 240 and each schematically show a state at a step. Constituents which are the same as those in FIGS. 12A and 12B are denoted with the same signs and will not be described.

First, on the substrate 243, the multilayer mirror 241a and the multilayer mirror 241b in each of which the high-refractive material and the low-refractive material are alternately stacked are formed (FIG. 13A), and on the sound matching material 244, the multilayer mirror 241b in which the high-refractive material and the low-refractive material are alternately stacked are formed (FIG. 13B). Here, the multilayer mirror 241b is formed by stacking layers using a masking technique so that the slit 246 is formed. It is preferable that the multilayer mirror 241a and the multilayer mirror 241b have the same properties, and in order to match the conditions such as variation in the thickness of each layer, the multilayer mirror 241a and the multilayer mirror 241b are preferably formed at the same time by the same sputtering device, for example.

Next, the spacer 245 is formed on a part of the multilayer mirror 241a formed on the substrate 243 (FIG. 13C). This spacer 245 can be formed by stacking layers using a masking technique by sputtering or the like. In the case of forming the spacer 245 by sputtering, a decrease in the thickness of the spacer 245 to around 30 nm will cause a slight decrease in the detection sensitivity, but allow a significant increase in the throughput.

At the end, the substrate 243 on which the spacer 245 and the multilayer mirror 241a are formed as described above and the sound matching material 244 on which the multilayer mirror 241b is formed as described above are disposed so that the multilayer mirror 241a and the multilayer mirror 241b face each other. Subsequently, the spacer 245 and the slit 256 are positioned so as to be at slightly different positions and are thus fixed (FIG. 13D).

Through the above process, the ultrasound detection element 240 having a large area can be relatively easily manufactured. Accordingly, the manufacture of the ultrasound detection element 240 by fabricating an element having a large area first and then cutting it at the end will produce an effect of enabling high-volume production.

It is to be noted that the above process is an example to which the present invention is not limited. It goes without saying that the effects of this embodiment will not change even when other manufacturing method is used.

As described above, in the ultrasound examination device 10 according to this embodiment, the ultrasound echoes can be detected with use of light as in the case of Embodiment 1, so that the receiving probe no longer needs the electrical connections, which allows an increase in the number of spots for receiving the ultrasound echoes and allows the spots to be disposed in a wide region. This produces effects of improving the S/N ratio and improving the resolution in the beam forming.

Furthermore, through appropriate setting, according to an elapsed time, of the wavelength and amount of the laser light 222 for detecting the ultrasound echoes 28, it is possible to detect even the ultrasound echoes 28 which have been largely attenuated. In addition, variation in the sensitivity of the ultrasound detection element 240 due to an error in the layer thickness or due to a surrounding environment can be reduced. Furthermore, the ultrasound detection element 240 has a simple structure in which the resonator structure is composed of a multilayer mirror of around ten layers on one side, and moreover, most of the ultrasound detection element 240 can be formed by sputtering, which results in the small number of steps in the manufacturing process and enables high throughput. This produces effects of enabling mass production at low cost and moreover giving an advantage of a reduction in the size of the receiving probe.

### Embodiment 3

Embodiment 2 has described that the optical probe which has the Fabry-Perot resonator structure and includes the ultrasound detection element having the sound matching material detects the ultrasound echoes with use of the deformation of the sound matching material caused by the ultrasound echoes. Embodiment 3 describes an example of the case where, although the optical probe has the Fabry-Perot resonator structure, the ultrasound detection element includes an acoustic lens, an acoustic mirror, or the like as the sound matching material.

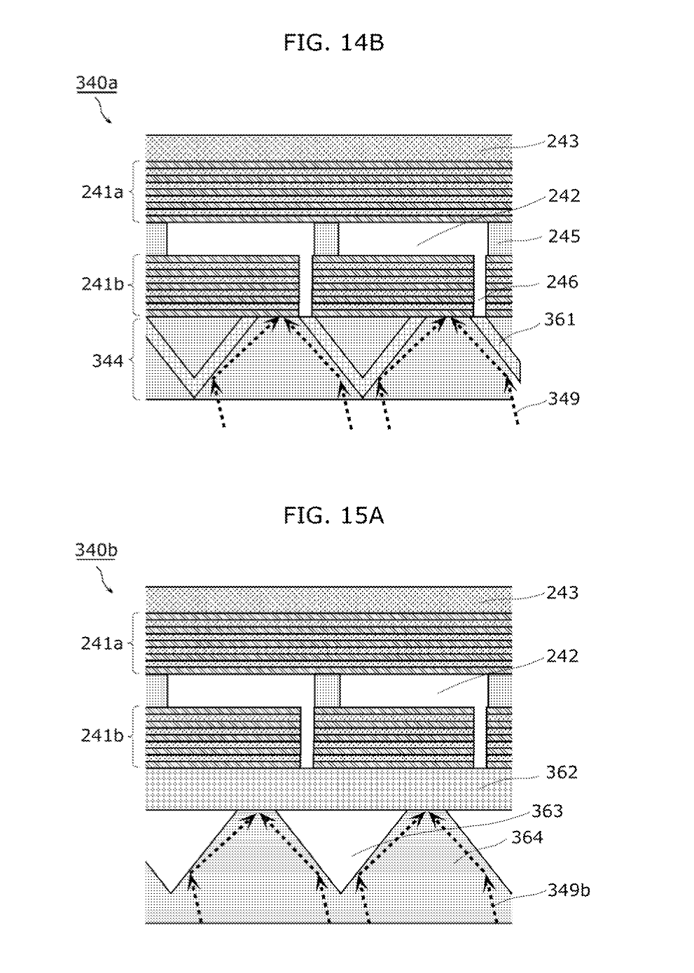

FIGS. 14A and 14B each illustrate a variation of a configuration of the ultrasound detection element in Embodiment 3 of the present invention. Constituents which are the same or like as those in FIG. 12A are denoted with the same signs and will not be described in detail.

FIG. 14A shows a configuration example which uses an acoustic lens 351, and FIG. 14B shows a configuration example which uses an acoustic mirror 361. In FIGS. 14A and 14B, the only difference from FIG. 12A is the acoustic lens 351 and the acoustic mirror 361, and the ultrasound echoes 249 are detected with their amplitude increased by the acoustic lens 351 and the acoustic mirror 361.

In an ultrasound detection element 340 shown in FIG. 14A, the multilayer mirror 241b is divided into a plurality of detection regions by the slits 246. For each of the detection regions, the acoustic lens 351 is placed inside the sound matching material 344.

When ultrasound echoes 349 enter the ultrasound detection element 340 configured as above, the acoustic lens 351 will converge the ultrasound echoes 349 for each of the detection regions and direct the converged ultrasound echoes 349 toward the multilayer mirror 241b. At this time, the amplitude of the ultrasound echoes 349 increases on the bottom of the multilayer mirror 241b, with the result that the variation range in thickness of the air layer 242 increases, which allows an improvement in the sensitivity for detecting the ultrasound echoes 349.

An ultrasound detection element 340a shown in FIG. 14B is configured using the acoustic mirror 361 instead of the acoustic lens 351. Even with this structure, it is possible to converge the ultrasound echoes 349 toward the bottom of the multilayer mirror 241b by reflecting the ultrasound echoes 349 on the acoustic mirror 361, which allows an increase in the amplitude of the ultrasound echoes 349. Thus, as in the case of the ultrasound detection element 340 shown in FIG. 14A, the detection sensitivity can be improved.

In the case of using the acoustic lens 351 in the ultrasound detection element as shown in FIG. 14A, the ultrasound echoes 349 are slightly but undesirably reflected on an interface of the acoustic lens 351. On the other hand, in the case of using the acoustic mirror 361 in the ultrasound detection element as shown in FIG. 14B, there is no such an energy loss, which allows a more effective improvement in the sensitivity.

Furthermore, there is a yet another conceivable variation of the configuration of the ultrasound detection element 340a shown in FIG. 14B. That variation is described next.

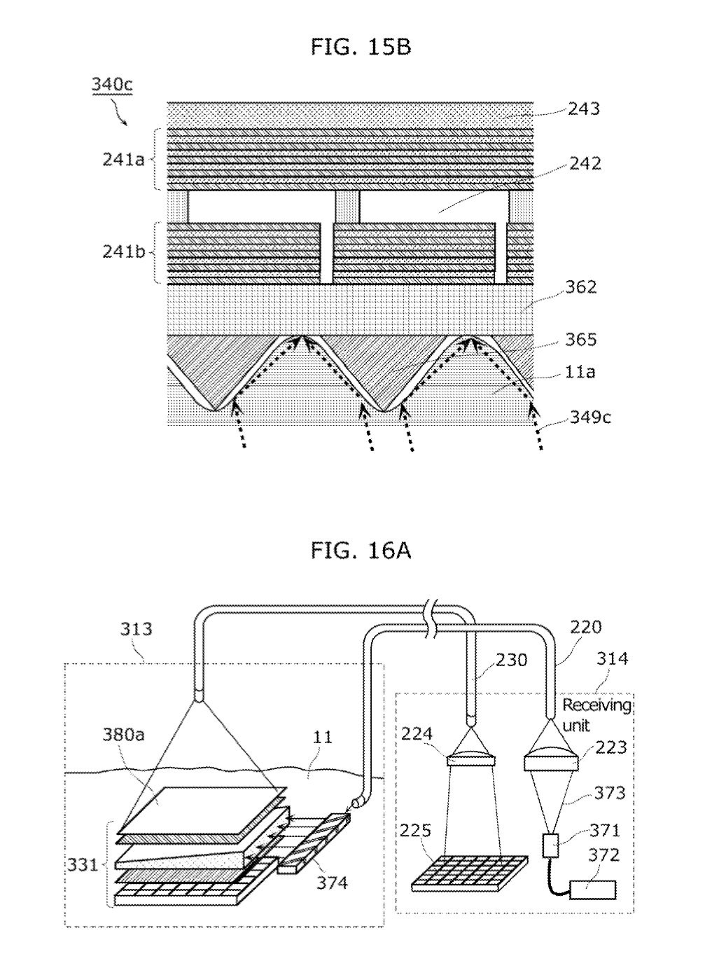

FIGS. 15A and 15B each illustrate another variation of the configuration of the ultrasound detection element in Embodiment 3 of the present invention. Constituents which are the same or like as those in FIG. 12A are denoted with the same signs and will not be described in detail.

FIG. 15A shows a configuration example which uses a wedge-shaped air layer, and FIG. 15B shows a configuration example in which the sound matching material has a wedge-shaped surface.

The ultrasound detection element 340b includes: a tapered member in form of a protrusion which has a cross-sectional area decreasing in a propagation direction of the ultrasound waves indicating vibration of a surface of the subject 11; and a mirror member located around the tapered member and having an acoustic impedance different from an acoustic impedance of the tapered member, and the ultrasound waves propagating inside the tapered member are reflected on the interface between the tapered member and the mirror member before reaching the first reflector element. Specifically, the ultrasound detection element 340b shown in FIG. 15A has a structure in which a thin film 362 and a sound matching material 364 having numerous protrusions are bonded to each other through the protrusions.

Each of the protrusions of the sound matching material 364 corresponds to a tapered member in an implementation of the present invention and is configured to have a cross-sectional area which becomes smaller toward the film 362. An air layer 363 corresponds to a mirror member in an implementation of the present invention and is formed to have a wedge shape.

Furthermore, the multilayer mirror 241b is formed on the film 362 and is divided so that the detection regions correspond to the protrusions of the sound matching material 364.

When ultrasound echoes 349b enter the sound matching material 364 of the ultrasound detection element 340b configured as above, the ultrasound echoes 349b are reflected on the boundary between the sound matching material 364 and the air layer 363. Thus, the ultrasound echoes 349b are converged for each of the detection regions, and the amplitude of the ultrasound echoes 349b is increased on the bottom of the multilayer mirror 241b. This allows an increase in the variation range in thickness of the air layer 242 and thereby allows an improvement in the sensitivity for detecting the ultrasound echoes 349b. Furthermore, with this structure, the air layer 363 forms a reflection surface, with the result that the sound matching material 364 is more likely to deform, which produces an effect of providing higher detection sensitivity.

Furthermore, it may also be possible that, as in an ultrasound detection element 340c shown in FIG. 15B, an acoustic mirror 365 disposed on a surface of the film 362 deforms the subject 11 so that the subject 11 forms a protrusion 11a. At this time, the gap between the subject 11 and the acoustic mirror 365 is desirably filled with a sound matching material which is liquid or gel that can deform to fill the gap.

Even with such a structure, ultrasound echoes 349c, can be converged and have increased amplitude, which allows an improvement in the detection sensitivity. Furthermore, with this structure, the amplified ultrasound echoes 349c propagate directly to the film 362, which produces an effect of loss reduction.

Although this embodiment describes, as an example, the configuration in which one spacer is provided for each of the detection regions, it is not always necessary to provide one spacer for every detection region. For example, it may be possible that a spacer is formed between the multilayer mirror 241a and the multilayer mirror 241b so as to fix one end of at least every other multilayer mirror 241b resulting from the division of the multilayer mirror 241b by the slits 246. In the detection region with no spacers among such detection regions each with or without the spacer, the ultrasound waves are desirably converged to propagate to the center or the center of gravity of each reflection region so that the second multilayer mirror is deformed, and the propagated ultrasound waves are amplified. With such a structure, when the ultrasound echoes propagate to the bottom of the multilayer mirror 241b, the reflection region has a reduced slope and in addition, the variation range in resonator length is wider, which allows an improvement in the detection sensitivity.

Furthermore, although each of the ultrasound detection elements 240, 340, 340a, 340b, and 340c in Embodiments has the air layer 242 as a resonator medium, gas other than air and liquid may be used, which produces the same or like effects.

Furthermore, the multilayer mirror 241a and the multilayer mirror 241b may each be a metal mirror, or a photonic crystal mirror configured to have a refractive index periodically changing in the reflection surface, or a subwavelength grating using a fine grating for a wavelength equal to or smaller than the wavelength of incident light, which produces the same or like effects.

### (Variation 1)

The above embodiments have described the case where the optical probe has the Fabry-Perot resonator structure. The following describes, as a variation, the case where the optical probe is composed of the heterodyne interference optical system.

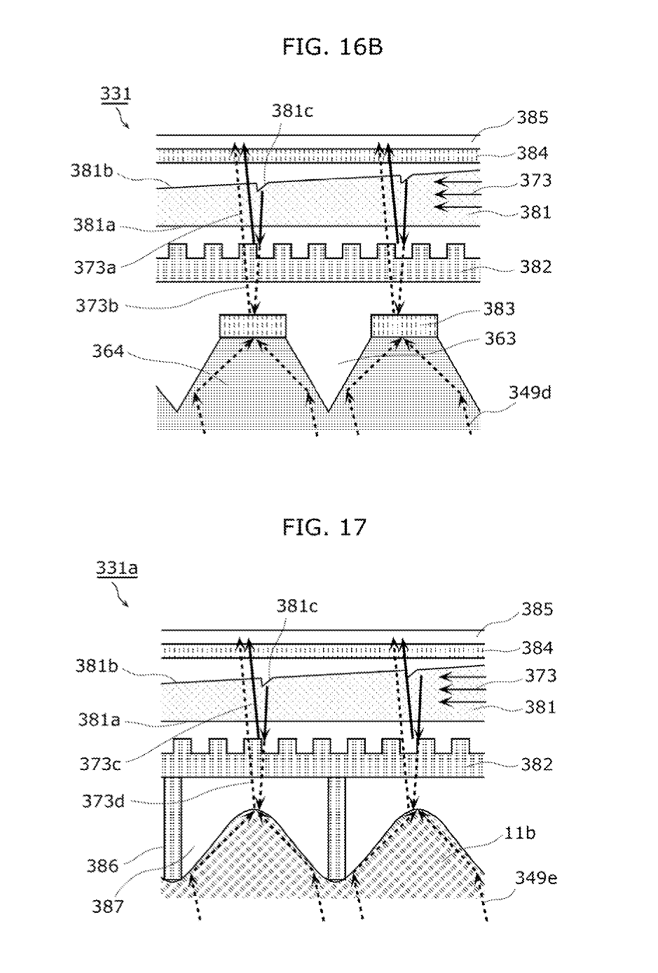

FIGS. 16A and 16B each show configurations of an optical probe and a receiving unit in Variation 1 of Embodiment 3 of the present invention. FIG. 16A shows schematic configurations of an optical probe 313 and a receiving unit 314, and FIG. 16B shows a schematic configuration of an ultrasound detection element 331. The only difference of this variation from the configuration of the ultrasound examination device in Embodiment 3 is the optical probe 313 and the receiving unit 314, and the other parts are the same and therefore will not be described. Constituents which are the same as those in FIGS. 11A and 11B are denoted with the same signs and will not be described.

The optical probe 313 and the receiving unit 314 in the ultrasound examination device 10 according to this variation are configured to detect, using the optical heterodyne technique, variation of the surface of the subject 11 caused by the ultrasound waves.

In FIG. 16A, the optical probe 313 and the receiving unit 314 are connected to each other with use of the optical fiber 220 and the image fiber 230 as in the configuration shown in FIG. 12. In this variation, the light source of the receiving unit 314 includes a semiconductor laser 371 and a current modulator 372 and is configured to superimpose a sawtooth current onto an injected current of the semiconductor laser 371 using the current modulator 372 and thereby emit laser light 373 generated by modulating the emitted light to have a sawtooth optical frequency. At a light-emitting end of the optical fiber 220, a gradient-index (GRIN) lens is disposed so as to collimate the light emitted from the optical fiber 220.

The optical probe 313 includes: a light guide rod 374 which converts, into linear parallel light, the laser light 373 sent through the optical fiber 220; and the ultrasound detection element 331. The optical probe 313 is configured to send, through the image fiber 230, an image of ripples of the ultrasound echoes appearing on a top surface 380a of the ultrasound detection element 331.

The ultrasound detection element 331 has a structure which includes the optical heterodyne interference system as shown in FIG. 16B, for example. Specifically, the ultrasound detection element 331 includes: a light guide plate 381 on whose side surface the laser light 373 emitted from the light guide rod 374 is incident and from whose main surface 381a the laser light 373 is emitted; a wire grid polarizer 382 disposed adjacent to the main surface 381a of the light guide plate 381; a reflector 383 which reflects light transmitted by the wire grid polarizer 382; a polarizer 384 disposed opposite to the wire grid polarizer 382 across the light guide plate 381; and a screen 385 on which an image is projected using light transmitted by the polarizer 384.

The light guide rod 374 has a structure which includes a plurality of deflection grooves each having an inclined surface inclined at approximately 45 degrees with respect to a side surface from which light is emitted, and totally reflects the light incident on the light guide rod 374 to deflect the light at approximately 90 degrees.

In an opposite surface 381b of the light guide plate 381, a plurality of deflection surfaces 381c are formed each of which is an inclined surface inclined at approximately 45 degrees with respect to the main surface 381a. The light guide plate 381 totally reflects the light incident in substantially parallel with the main surface 381a, to deflect the light toward the main surface 381a, and then emits the light substantially vertically from the main surface 381a.

The reflector 383 is disposed on the top surface of a protrusion of the sound matching material 364 having the numerous protrusions and is configured so that each of the protrusions becomes the detection region.

The wire grid polarizer 382 and the polarizer 384 are configured to have transmission axes which are different from each other by approximately 45 degrees. The polarizer 384 is configured to transmit part of laser light 373a reflected by the wire grid polarizer 382 and part of laser light 373b transmitted by the wire grid polarizer 382 and reflected by the reflector 383.

Next, the ultrasound detection operation in this variation configured as above is described with reference to FIGS. 16A and 16B.

First, the semiconductor laser 371 emits the laser light 373 with an optical frequency modulated by the current modulator 372. This laser light 373 is provided to the optical probe 313 via the condensing lens 223 and the optical fiber 220. The provided laser light 373 is collimated by the GRIN lens (not shown) and emitted from the optical fiber 220, and then converted into linear parallel light by the light guide rod 374 before entering the ultrasound detection element 380. The laser light 373 incident on the light guide plate 381 of the ultrasound detection element 331 is deflected by the deflection surface 381c and emitted substantially vertically from the main surface 381a of the light guide plate 381, and then partially transmitted and partially reflected by the wire grid polarizer 382.

The laser light 373a reflected by the wire grid polarizer 382 passes through the light guide plate 381 and enters the polarizer 384 while the laser light 373b transmitted by the wire grid polarizer 382 is reflected by the reflector 383 and passes through the wire grid polarizer 382 and the light guide plate 381 again to enter the polarizer 384.

At this time, the transfer of ultrasound echoes 349d from the subject 11 to the sound matching material 364 will cause, for each detection region, the ultrasound echoes 349d to be converged by the protrusion of the sound matching material 364, resulting in an increase in the amplitude of the ultrasound echoes 349d which vibrate the reflector 383. Accordingly, the laser light 373b reflected by the reflector 383 will have a slightly shifted optical frequency due to the Doppler shift.

The polarized light of the laser light 373a arrived at the polarizer 384 is orthogonal to the polarized light of the laser light 373b arrived at the polarizer 384, and the polarizer 384 is designed to have a transmission axis at approximately 45 degrees with the polarized light. Thus, either one of the laser light is partially transmitted by the polarizer 384, and the transmitted light is then multiplexed. The polarized light of the multiplexed laser light 373a and the polarized light of the multiplexed laser light 373b are the same and therefore superimposed as coherent light on the screen 385. Intensity distribution of this coherent light is sent to the receiving unit 314 through the image fiber 230 and magnified by the magnifying lens 224 and then received as two-dimensional intensity distribution by the light detector 225.

Here, the intensity of the coherent light in a certain region, which is observed on the screen 385, leads to a beat signal which corresponds to a difference in optical path length between the laser light 373a and the laser light 373b, and vibration of the reflector 383 will therefore appear as a frequency shift of the beat signal. Accordingly, it is possible to detect an echo signal in a two-dimensional plane by demodulating a beat signal which has been frequency-modulated from a signal detected by the light detector 225.

A structure of an optical heterodyne technique, adopted in a laser displacement meter or the like, is a structure of separating the reference light inside a measurement device and therefore has a problem of S/N ratio deterioration which is due to addition of noise generated by vibration between the measurement device and an object to be measured to information on displacement of the object itself. In contrast, the structure in this variation is a structure of detecting relative displacement or vibration with respect to the wire grid polarizer 382 and the reflector 383, which produces an effect of reducing the noise which is generated by vibration in a surrounding environment.

As above, even with the structure of detecting the ultrasound waves, the ultrasound echoes can be detected with use of light, with the result that the receiving probe no longer needs the electrical connections, which allows an increase in the number of spots for receiving the ultrasound echoes and allows the spots to be disposed in a wide region. This makes it possible to improve the S/N ratio and improve the resolution in the beam forming. Furthermore, the ultrasound detection element 331 requires less accuracy for the wavelength band of the light source, manufacturing variations, and the like, which results in high productivity.

Since the ultrasound detection elements 240, 340, 340a, 340b, 340c, and 331 in this variation use no mechanical resonance, the detection is possible regardless of the frequency of the ultrasound waves. It is therefore possible to use each of the ultrasound detection elements in combination with an ultrasound probe (transmitting probe) of a plurality of frequencies according to applications. Thus, for a user who uses a transmitting probe of different frequencies, the cost for probes can be reduced.

### (Variation 2)

The ultrasound detection element 331 shown in FIG. 16B includes the sound matching material 364 and the reflector 383 so as to generate the beat signal which includes information on vibration of the ultrasound echoes 394d, the present invention is not limited to the above. Since it is sufficient that the beat signal which includes the information on vibration of the ultrasound echoes can be generated, another configuration is also conceivable which does not include the sound matching material 364 and the reflector 383. Such an example is described below as Variation 2.

FIG. 17 schematically shows a configuration of an ultrasound detection element in Variation 2 of Embodiment 3 of the present invention. Constituents which are the same or like as those in FIG. 16B are denoted with the same signs and will not be described in detail. An ultrasound detection element 331a shown in FIG. 17 is different from the ultrasound detection element 331 in the above Variation 1 in that the sound matching material 364 and the reflector 383 are not provided while a rod 386 is provided. The other parts are the same and therefore not described.

Since it is sufficient that the ultrasound detection element 331a shown in FIG. 17 can generate a beat signal which includes information on vibration of ultrasound echoes 349e, the rod 386 deforms the subject 11 so that the subject 11 forms a protrusion 11b. At this time, the gap between the subject 11 and the rod 386 is an air layer 387. Since this air layer 387 is a gap (an air layer) formed when the subject 11 is deformed by the rod 386, the gap may be filled with a sound matching material which is liquid or gel.

Even with such a structure, the ultrasound echoes 349e can be converged and have increased amplitude by the protrusion 11b of the subject 11 deformed by the rod 386, which produces an effect of improving the detection sensitivity.

### Embodiment 4

Embodiments 1 to 3 have described that the ultrasound probe 12 is composed of a transducer array in which a plurality of transducers are arranged in two dimensions and each of the transducers is constituted by forming an electrode on a piezoelectric element made of, for example, piezoelectric ceramic represented by lead zirconate titanate (PZT), but the present invention is not limited to the above and may be implemented with another configuration. In Embodiment 4, a configuration different from that of the ultrasound probe 12 in Embodiments 1 to 3 is described.

Since the ultrasound probe 12 is composed of the transducer array in which the plurality of transducers are arranged in two dimensions, the ultrasound probe 12 needs to highly integrate a larger number of transducers, which poses a difficulty growing in size for the reasons of microfabrication of the piezoelectric elements included in the transducers, a difficulty in the electrical connections to the large number of piezoelectric elements, and so on. Even when the ultrasound probe 12 succeeds in growing in size, the number of transducers will be enormous, which may cause a problem of cost, power consumption, or the like, of the system. For such reasons, the ultrasound probe 12 has a difficulty in growing in size, resulting in a difficulty in examining a wide range.

In response, the ultrasound probe 12 which uses only effective ones of the transducers by adequate selection, that is, of what is called a sparse type, has been proposed. The ultrasound probe 120f this sparse type is capable of performing sector scanning on a wide region with a reduced number of transducers owing to the selection, but is not capable of examining, even with an increased scan angle, a region other than the region around the probe in the case of examining a narrow region. This means that the ultrasound probe 12 of the sparse type is suited to, for example, a cardiac examination, which includes transmitting ultrasound waves from gaps of ribs, but is not suited to an application, such as a breast or abdominal examination, which is a broad examination from a narrow region to a deep region.

Thus, this embodiment describes an ultrasound probe and an ultrasound examination device including the ultrasound probe, which can be easily manufactured with a reduced number of transducers that are used to transmit ultrasound waves and need to be highly integrated and which is capable of high-speed three-dimensional scanning and a broad examination from a narrow region to a deep region.

The ultrasound examination device in this embodiment is the same or like as the ultrasound examination device 10 shown in FIG. 1 and therefore is not described. In order to describe a characteristic configuration of the ultrasound probe, the ultrasound probe 12 shown in FIG. 1A is described as an ultrasound probe 12a. Furthermore, in order to simplify descriptions, the optical probe 13 is described below as a conventional receiving probe 913.

The ultrasound probe 12a includes a group of transducers which are capable of two-dimensionally or three-dimensionally transmitting ultrasound waves. The transducer group 112 includes a plurality of transducers (a group of transducers) arranged in two dimensions, and each of the transducers is constituted by forming an electrode on a piezoelectric element made of, for example, piezoelectric ceramic represented by lead zirconate titanate (PZT). This transducer group 122 is configured to not only generate ultrasound pulses but also focus and deflect the generated ultrasound waves by applying, to the electrode of each of the transducers, pulsed voltage processed with a delay and transmitted from the transmitting unit 18. This structure allows the transducer group 122 to transmit the ultrasound waves 26 in a three-dimensional direction. Furthermore, with a plurality of transducer groups 122, the ultrasound probe 12a is configured to transmit the ultrasound waves 26 in a wide range.

The receiving probe 913 includes a detection element group as 132 which has a plurality of detection elements, and each of the detection elements has, for example, a structure obtained by forming an electrode on a piezoelectric element and is configured to have the same resonance frequency as that of the transducer group 122. With this structure, propagation of the ultrasound echoes 28 of the same frequency as the ultrasound waves 26 transmitted from the transducer group 122 to the detection element group 132 causes each piezoelectric element of the detection element to resonate, with the result that voltage is generated by the piezoelectric effect of each piezoelectric element. Each of the detection elements is configured to detect the ultrasound waves 28 by detecting a change in this voltage. As in the above-described case, the reflector 43 (not shown) for transferring displacement (vibration) of a surface of the subject 11 caused by the ultrasound waves 26 may be disposed between the receiving probe 913 and the subject 11.

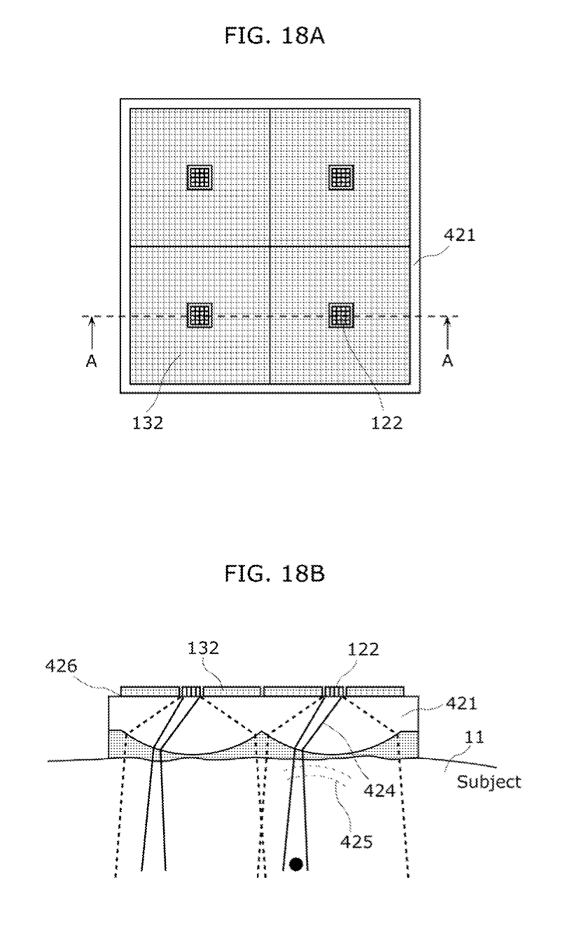

FIGS. 18A and 18B each schematically show a configuration in which the transducer group and the detection element group are arranged in Embodiment 4 of the present invention. FIG. 18A is a top plan view, and FIG. 18B is a cross-sectional view taken along A-A of FIG. 18A. As shown in FIG. 18A, the plurality of transducer groups 122 are arranged on the subject 11, and around each of the transducer groups 122, the detection element group 132 is disposed. Furthermore, as shown in FIG. 18B, an acoustic lens 421 corresponding to the transducer group 122 is disposed between (i) the transducer group 122 and the detection element group 132 and (ii) the subject 11.

The acoustic lens 421 corresponds to an ultrasound deflecting element in an implementation of the present invention and deflects the ultrasound waves transmitted by the ultrasound probe 12a so that the ultrasound waves substantially vertically enter the subject 11. Specifically, the acoustic lens 421 is configured to cause ultrasound waves 424 transmitted from the transducer group 122 of the ultrasound probe 12a, to substantially vertically enter the subject 11.

In the case where the reflector 43 is provided, it is sufficient that the acoustic lens 421 is formed integrally with the reflector 43 and is located between the reflector 43 and the subject 11 and that the reflector 43 is disposed in close contact with the subject 11 via the acoustic lens 421.

Next, the operation of the ultrasound examination device according to this embodiment configured as above is specifically described.

First, the control unit 19 controls the transmitting unit 18 so that the transmitting unit 18 generates the drive signal with predetermined timing, and the transmitting unit 18 performs delay processing for focusing and deflecting the ultrasound waves and supplies drive pulses processed with a delay to each of the plurality of transducer groups 122 of the ultrasound probe 12a. According to the drive pulses transmitted from the transmitting unit 18, the ultrasound probe 12a transmits the ultrasound waves 424 in a predetermined direction from each of the transducer groups 122.

Next, the ultrasound waves 424 transmitted from the transducer group 122 are deflected by the acoustic lens 421 shown in FIG. 18B and are substantially vertically incident on the subject 11. The incident ultrasound waves 424 are reflected on an interface of tissue having different acoustic impedances inside the subject 11 and propagate, as ultrasound echoes 425, to the surface of the subject 11 and are deflected again by the acoustic lens 421 before arriving at a transmitting/receiving surface 426. The ultrasound echoes 425 arrived at the transmitting/receiving surface 426 cause the piezoelectric element of each detection element of the detection element group 132 to mechanically resonate, and from a change in the voltage generated by the piezoelectric effect, an echo signal is detected.

This echo signal detection operation is performed while changing the transmission direction of the ultrasound waves 424 which are transmitted from the transducer group 122. Here, dotted lines in FIG. 18B indicate scan ranges of the ultrasound waves 424 and as indicated by these dotted lines, this scanning is sector scanning at the time when the ultrasound waves 424 are transmitted from the transducer group 122. However, the ultrasound waves 424 used in this sector scanning are deflected by the acoustic lens 421 so as to substantially vertically enter the subject, with the result that the scanning inside the subject will be like linear scanning.

Such scanning produces an effect of expanding the scan range in a narrow part of the subject 11 as compared to the ordinary sector scanning. Furthermore, since this configuration can expand the examination region using the plurality of transducer groups 122, not only a gap between the examination regions can be removed, but also an overlap of the examination regions can be reduced, which produces an effect of improving efficiency of the examination.

Conventionally, for such wide-range two-dimensional linear scanning, the transducers need to be densely arranged in a region having the same size as the region to be scanned, which are difficult to achieve for the reasons of microfabrication of the piezoelectric elements, a difficulty in the electrical connections, and so on. In the configuration according to this embodiment, only the transducer group 122 requires such dense arrangement of the transducers while the detection element group 132 which likewise includes the piezoelectric elements does not necessarily require such dense arrangement of detection elements (the piezoelectric elements). It is therefore possible to decrease the level of difficulty in manufacture, allowing facilitated manufacture.

The detection element group 132 may not only have the detection elements less densely arranged, but also have a sparse array in which the detection elements are randomly distributed. With such a structure, an amount of computation and memory for processing detection data can be reduced, which allows a reduction in cost and power consumption.

The following continues to describe the operation of the ultrasound examination device. The echo signal detected by the receiving probe 913 is amplified and digitally converted by the receiving unit 14 and sent to the signal processing unit 15. In the signal processing unit 15, first, this echo signal is stored in the storing unit 15a, and on the basis of the signal data stored in the storing unit 15a, the arithmetic processing unit 15b performs the phasing addition, that is, the beam forming processing, and obtained three-dimensional data are stored in the image memory 15c.

The above operation is performed while scanning the inside of the subject 11, and information on the entire examined region is computed and stored in the image memory 15c. The three-dimensional data stored in the image memory 15c are rendered as a three-dimensional image by the image processing unit 16, and the resultant image is displayed by the image display unit 17. Through the operation as above, an ultrasound image in a wide range can be obtained.

As above, the ultrasound examination device according to this embodiment is capable of examining a broader region by using the plurality of transducer groups 122. Furthermore, since the transducer groups 122 can be arranged so that there is no gap between the respective scan ranges while an overlap between the respective scan ranges is small, the examination can be efficiently performed, that is, the broad examination in a short time becomes possible. As compared to the linear scanning for a comparable examination region, the number of transducers to be densely arranged can be significantly reduced, which mitigates the problems related to microfabrication of the piezoelectric elements, the electrical connections, and so on, allowing facilitated manufacture. Furthermore, since the detection element group 132 can be formed separately from the transducer group 122 of the ultrasound probe 12, the openings of the receiving probe 913 can be larger. This makes it possible to improve the resolution in the beam forming.

Although the above has described that the ultrasound examination device according to this embodiment includes the acoustic lens 421 so as to reduce the gap and overlap of the respective scan ranges of the transducer groups 122 (the ultrasound probe 12a), the present invention is not limited to the above. The ultrasound examination device may include an acoustic coupler (wedge), which produces the same or like effect.

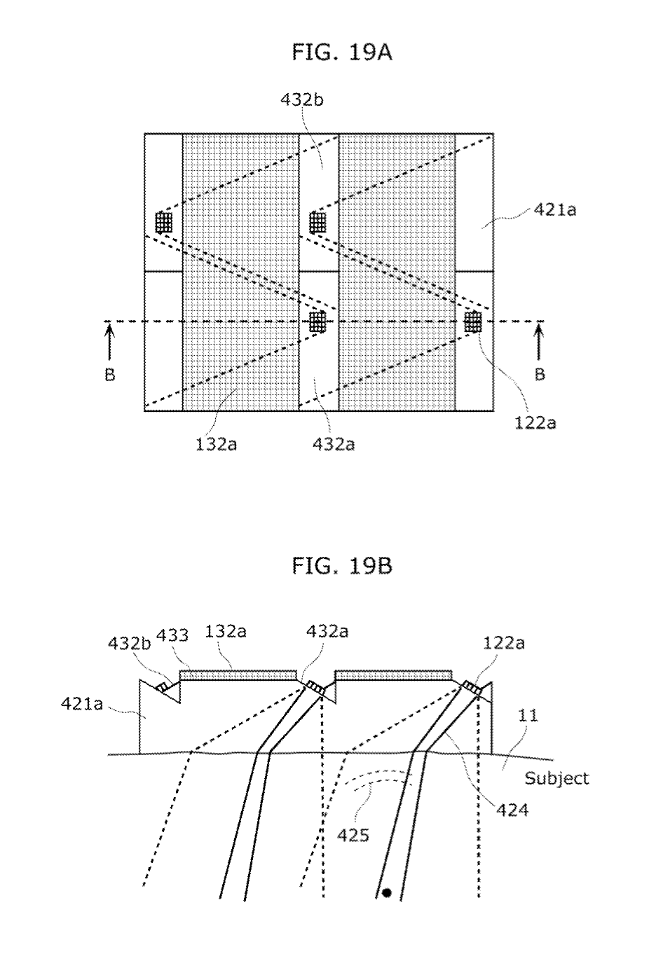

FIGS. 19A and 19B each schematically show another configuration in which the transducer group and the detection element group are arranged in Embodiment 4 of the present invention. FIG. 19A is a top plan view, and FIG. 19B is a cross-sectional view taken along B-B of FIG. 19A. The configuration of FIGS. 19A and 19B is different from the configuration of FIGS. 18A and 18B only in that an acoustic coupler 421a is provided instead of the acoustic lens 421. Constituents which are the same or like as those in FIGS. 18A and 18B are denoted with the same signs and will not be described.

As shown in FIGS. 19A and 19B, the acoustic coupler 421a is disposed between (i) a transducer group 122a and a detection element group 132a and (ii) the subject 11.

The acoustic coupler 421a corresponds to an ultrasound deflecting element in an implementation of the present invention and deflects the ultrasound waves transmitted by the ultrasound probe 12a so that the ultrasound waves substantially vertically enter the subject 11. Furthermore, the acoustic coupler 421a has a surface inclined with respect to a surface of the subject 11, and on the inclined surface, the transducer group 122a of the ultrasound probe 12a is disposed.

Specifically, the acoustic coupler 421a has inclined surfaces 432a and 432b and a horizontal surface 433, with respect to the surface of the subject 11. On each of the inclined surface 432a and 432b, the transducer group 122a is disposed, and on the horizontal surface 433, the detection element group 132a is disposed. With this structure, the ultrasound waves 424 are obliquely incident on the subject 11. The acoustic coupler 321a is designed to have an acoustic impedance such that, upon entering the subject 11, the obliquely-incident ultrasound waves 424 are refracted and thereby deflected in a substantially vertical direction.

The inclined surface 432a and the inclined surface 432b are inclined in opposite directions and configured so that the transmission directions of the respective ultrasound waves 424 face each other as indicated by dotted lines in FIG. 19A and that the respective scan ranges overlap without gaps.

The acoustic coupler 421a and the transducer group 122a configured as above perform sector scanning at the time when the ultrasound waves 424 are transmitted from the transducer group 122a. However, because of the refraction between the acoustic coupler 421a and the subject 11 as well as the arrangement of the transducer groups 122a, the arranged transducer groups can be such that there is no gap between the respective scan ranges while an overlap between the respective scan ranges is small, which allows an efficient examination that makes the broad examination in a short time possible. In addition, since only the transducer group 122a requires the dense formation of transducers, the manufacture is facilitated.

Furthermore, in the configuration in which the ultrasound examination device includes the acoustic lens 421 as shown in FIG. 18B, the ultrasound echoes 25 are slightly but undesirably reflected on an interface of the acoustic lens 421. On the other hand, in the configuration in which the ultrasound examination device includes the acoustic coupler 421a, there is no energy loss, which produces a further effect of improving the sensitivity for detecting the ultrasound echoes 425.

Although this embodiment describes the configuration in which the transmission and reception of the ultrasound waves are performed by the piezoelectric elements, the present invention is not limited to the configuration. For example, a capacitive micromachined ultrasonic transducer (hereinafter referred to as CMUT) using micromachining technique may be used. In the case of using the piezoelectric elements, the sensitivity may decrease due to a difference in resonance frequency between the transducer group and the detection element group, but the CMUT has small property variations and therefore is less likely to have decreasing sensitivity attributed to the difference in resonance frequency.

### (Variation 1)

The above describes the configuration in which the reception of the ultrasound waves is performed by the piezoelectric elements, but the present invention is not limited to the configuration. As described in Embodiments 1 to 3, the configuration of detecting the ultrasound waves with use of light may be employed. The following Variation 1 describes the configuration in which, instead of the as receiving unit 14 and the receiving probe 913, the receiving unit 314 for detecting the ultrasound waves with use of light, and the optical probe 413 are provided.

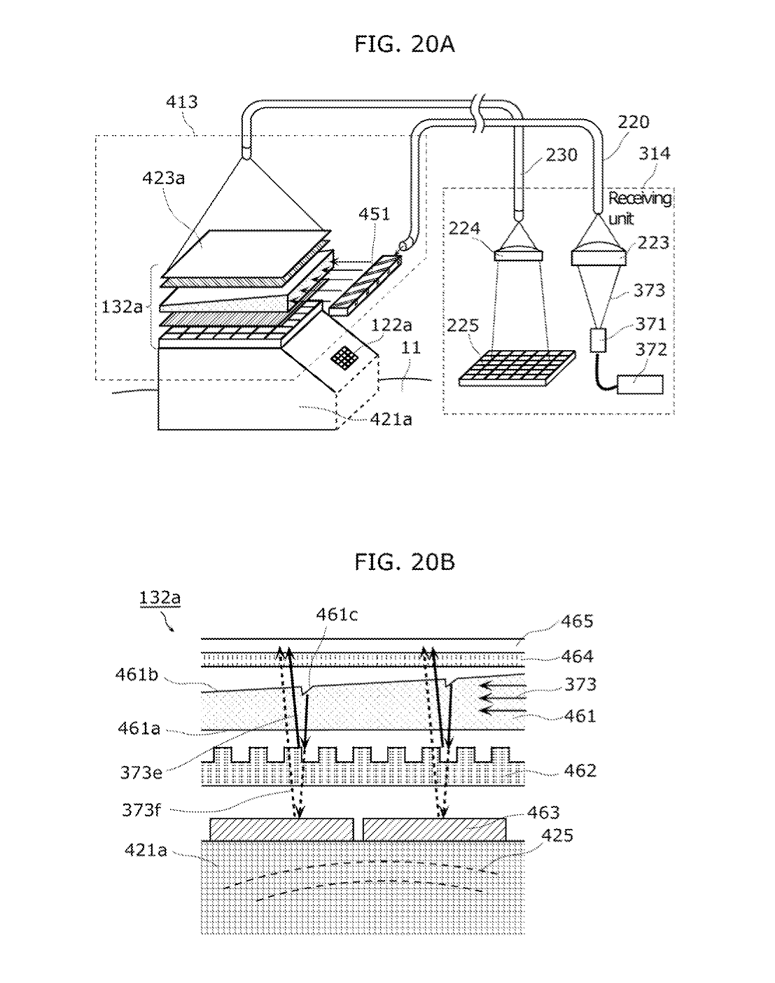

FIGS. 20A and 20B each show a configuration of an ultrasound examination device in Variation 1 of Embodiment 4 of the present invention. Constituents which are the same as those in FIG. 16A are denoted with the same signs and will not be described. FIG. 20A schematically shows configurations of the optical probe 413 and the receiving unit 314, and FIG. 20B schematically shows the detection element group 132a. The only difference of the configuration of this variation from the configuration of Embodiment 4 is the optical probe 413 and the receiving unit 314, and the other parts are the same and therefore will not be described.

The optical probe 413 and the receiving unit 314 in the ultrasound examination device according to this variation are configured to detect, using the optical heterodyne technique, the vibration caused by the ultrasound waves propagated from the subject 11 to the ultrasound coupler 421a shown in FIGS. 19A and 19B. The receiving unit 314 is configured to supply the optical probe 413 with appropriate light for detecting the ultrasound echoes and to convert, into an electric signal, the light output from the optical probe 413, and then output the electric signal.

In FIG. 20A, the optical probe 413 and the receiving unit 214 are connected to each other with use of the optical fiber 220 and the image fiber 230 in which several tens of thousands of optical fibers are placed so that an image can be conveyed. The light source of the receiving unit 414 includes the semiconductor laser 371 and the current modulator 372 and is configured to superimpose a sawtooth current onto an injected current of the semiconductor laser 371 using the current modulator 372 and thereby emit the laser light 373 generated by modulating the emitted light to have a sawtooth optical frequency. Furthermore, in the configuration, the condensing lens 223 is provided which collects the laser light 373 emitted from the semiconductor laser 371, and the laser light 373 is sent through the optical fiber 220. Furthermore, the configuration includes: the magnifying lens 224 which magnifies the image sent through the image fiber 230; and the light detector 225 having a two-dimensional array composed of a charge-coupled device (CCD), a metal oxide semiconductor (MOS) sensor, or a plurality of photodiodes (PD), for receiving light of the magnified image.

At a light-emitting end of the optical fiber 220, a gradient-index (GRIN) lens is disposed so as to collimate the light emitted from the optical fiber 220. The optical probe 413 includes: a light guide rod 451 which converts, into linear parallel light, the laser light 373 sent through the optical fiber 220; and the detection element group 132a, and sends, through the image fiber 230, an image of ripples of the ultrasound echoes appearing on a top surface 423a of the detection element group 132a.

The detection element group 132a has a structure shown in FIG. 20B, for example. Specifically, the detection element group 132 includes: a light guide plate 461 on whose side surface laser light 443 emitted from the light guide rod 451 is incident and from whose main surface 461a the laser light 443 is emitted; a wire grid polarizer 462 disposed adjacent to the main surface 461a of the light guide plate 461; a plurality of divided reflectors 463 which reflects light transmitted by the wire grid polarizer 462; a polarizer 464 disposed opposite to the wire grid polarizer 462 across the light guide plate 461; and a screen 465 on which an image is projected using light transmitted by the polarizer 464.

The light guide rod 451 has a structure which includes a plurality of deflection grooves each having an inclined surface inclined at approximately 45 degrees with respect to a side surface from which light is emitted, and totally reflects the light incident on the light guide rod 451 to deflect the light at approximately 90 degrees.

In an opposite surface 461b of the light guide plate 461, a plurality of deflection surfaces 461c are formed each of which is an inclined surface inclined at approximately 45 degrees with respect to the main surface 461a so as to totally reflect the light incident in substantially parallel with the main surface 461a, to deflect the light toward the main surface 461a, and then emit the light substantially vertically from the main surface 461a.

The wire grid polarizer 462 and the polarizer 464 are configured to have transmission axes which are different from each other by approximately 45 degrees. The polarizer 464 is configured to transmit part of laser light 443a reflected by the wire grid polarizer 462 and part of laser light 443b transmitted by the wire grid polarizer 462 and reflected by the reflector 463.

Next, the ultrasound detection operation in this variation configured as above is described with reference to FIGS. 20A and 20B.

First, the semiconductor laser 371 emits the laser light 373 with an optical frequency modulated by the current modulator 372. This laser light 373 is provided to the optical probe 413 via the condensing lens 223 and the optical fiber 220. The provided laser light 373 is collimated by the GRIN lens (not shown) and emitted from the optical fiber 220, and then converted into linear parallel light by the light guide rod 451 before entering the detection element group 132a. The laser light 373 incident on the light guide plate 461 of the detection element group 132a is deflected by the deflection surface 461c and emitted substantially vertically from the main surface 461a of the light guide plate 461, and then partially transmitted and partially reflected by the wire grid polarizer 462.

Laser light 373e reflected by the wire grid polarizer 462 passes through the light guide plate 461 and enters the polarizer 464 while laser light 373f transmitted by the wire grid polarizer 462 is reflected by the reflector 463 and passes through the wire grid polarizer 462 and the light guide plate 461 again to enter the polarizer 464.

At this time, the transfer of ultrasound echoes 425 to the top surface of the acoustic coupler 421a vibrates the reflector 463, and the laser light 373f reflected by the reflector 463 will have a slightly shifted optical frequency due to the Doppler shift.

The polarized light of the laser light 373e arrived at the polarizer 464 is orthogonal to the polarized light of the laser light 373f arrived at the polarizer 464, and the polarizer 464 is designed to have a transmission axis at approximately 45 degrees with the polarized light. Thus, either one of the laser light is partially transmitted by the polarizer 464, and the transmitted light is then multiplexed. The polarized light of the multiplexed laser light 373e and the polarized light of the multiplexed laser light 373f are the same and therefore superimposed as coherent light on the screen 465. Intensity distribution of this coherent light is sent to the receiving unit 414 through the optical fiber 220 and magnified by the magnifying lens 224 and then received as two-dimensional intensity distribution by the light detector 225.

Here, the intensity of the coherent light in a certain region, which is observed on the screen 465, leads to a beat signal which corresponds to a difference in optical path length between the laser light 373e and the laser light 373f, and vibration of the reflector 463 will therefore appear as a frequency shift of the beat signal. Accordingly, it is possible to detect an echo signal in a two-dimensional plane by demodulating a beat signal which has been frequency-modulated from a signal detected by the light detector 225.

As above, the ultrasound examination device in this variation is configured to optically detect the ultrasound echoes 425. With such a structure, there is no longer need for microfabrication of the piezoelectric elements and electrical connections in the optical probe 413, with the result that the receiving spots of the ultrasound echoes 425 can be increased and can also be arranged in a wide region. This makes it possible to improve the S/N ratio and improve the resolution in the beam forming.

The configuration according to this variation for detecting ultrasound waves by using the optical heterodyne technique may include an acoustic lens. This is described below as Variation 2.

### (Variation 2)

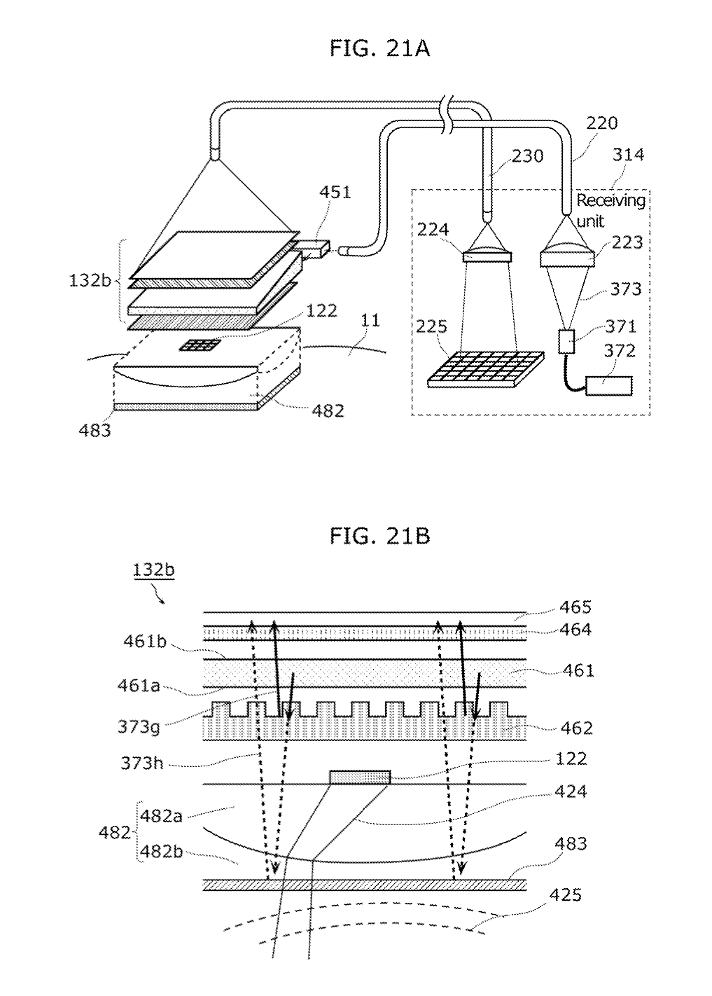

FIGS. 21A and 21B each show a configuration of an ultrasound examination device in Variation 2 of Embodiment 4 of the present invention. Constituents which are the same or like as those in FIGS. 20A and 20B are denoted with the same signs and will not be described. FIG. 21A shows schematic configurations of the receiving unit 314 and an optical probe 413a including the transducer group 122, and FIG. 21B shows a schematic configuration of the optical probe 413a including the transducer group 122. The only difference in FIGS. 21A and 21B from FIGS. 20A and 20B is an acoustic lens 482 and the optical probe 413a including the transducer group 122, and the other parts are the same and therefore will not be described. Constituents which are the same as those in FIGS. 20A and 20B are denoted with the same signs and will not be described.

As shown in FIG. 21A, the optical probe 413a includes a detection element group 132b, the acoustic lens 482, and the transducer group 122 and is configured such that the laser light 373 emitted from the light guide rod 451 is incident and an image of in ripples of ultrasound echoes appearing on the top surface of the detection element group 132b is sent through the image fiber 230.

The detection element group 132b includes, as shown in FIG. 21B, the light guide plate 461, the wire grid polarizer 462, the polarizer 464, and the screen 465 which have the same or like structures as those shown in FIG. 20B, for example.

The acoustic lens 482 corresponds to an ultrasound deflecting element in an implementation of the present invention and includes a combination of transmissive members of two kinds which have equal optical refractive indices and different acoustic impedances. Specifically, the acoustic lens 482 is composed of a transmissive member 482a and a transmissive member 482b which are of two kinds with equal optical refractive indices and different acoustic impedances, and refracts the ultrasound waves 424, but does not refract laser light 373h. Furthermore, a reflection layer 483 is disposed between the acoustic lens 482 and the subject 11 and is configured to vibrate according to the ultrasound echoes 425 propagating from the subject 11 and reflect the laser light 373h. On the top surface of the acoustic lens 482, the transducer group 122 is disposed.

The detection element group 132b configured as above is capable of performing scanning such as linear scanning owing to the acoustic lens 482 and is also capable of substantially vertically transmitting the ultrasound waves 424 to the subject 11, as in the structure shown in FIGS. 18A and 18B. With this structure, combining a plurality of transmitting/receiving elements allows efficient broad examination in a short time.

Furthermore, as in the case of the structure shown in FIGS. 20A and 20B, the vibration caused by the ultrasound echoes 425 propagated from the subject 11 is transferred to the reflection layer 483, and this information is given to the laser light 373h which is reflected on the reflection layer 483, so that the ultrasound echoes 425 can be detected. With this structure, there is no longer need for microfabrication of the piezoelectric elements and electrical connections, with the result that the receiving spots of the ultrasound echoes 425 can be increased and can also be arranged in a wide region. This makes it possible to improve the S/N ratio and improve the resolution in the beam forming.

In this structure, since the reflection layer 483 is further disposed between the acoustic lens 482 and the subject 11, the ultrasound echoes 425 propagated from the inside of the subject 11 can be attenuated by the acoustic lens 482 or, before reflection, can give a signal (vibration) to the laser light 373h. This allows an improvement in the sensitivity for detecting the echo signal.

As above, according to the present invention, it is possible to provide an ultrasound probe which is capable of structurally improving the resolution and to provide an ultrasound examination device using the ultrasound probe.

Specifically, the ultrasound probe and the ultrasound examination device using the ultrasound probe according to an implementation of the present invention produce an effect of providing high resolution and reducing the length of shooting time. More specifically, according to the present configuration, the reflected waves (ultrasound waves) from the subject can be detected by the large openings, with the result that high resolution can be obtained as compared to the width of ultrasound waves to be transmitted. Furthermore, not only the width of ultrasonic waves to be transmitted but also a scanning pitch can be increased, with the result that the number of times to transmit and receive the ultrasound waves in scanning can be smaller, which allows a decrease in the length of shooting time. Furthermore, with the structure of detecting the ultrasound echoes with use of light, it is possible to provide the structure in which a large number of receiving spots are arranged in two dimensions.

Although the ultrasound probe and the ultrasound examination device using the ultrasound probe according to implementations of the present invention have been described above based on the embodiments, the present invention is not limited to these embodiments. The scope of the present invention includes other embodiments that are obtained by making various modifications that those skilled in the art could think of, to the present embodiments, or by combining constituents in different embodiments. This means that the configurations shown in Embodiments 1 to 4 of the present invention are an example and may be changed in various ways within the scope of the present invention. Furthermore, all the configurations may be combined in any way, and it goes without saying that each of the configurations produces the effects inherent to the present invention.

### INDUSTRIAL APPLICABILITY

The present invention is applicable to ultrasound probes and ultrasound examination devices using the same and particularly applicable to an ultrasound probe which demands high-speed three-dimensional scanning as well as an increase in the examination region, and also applicable to an ultrasound examination device using the ultrasound probe.

What is claimed is:

1. An ultrasound probe comprising:
an ultrasound transmitting unit configured to transmit ultrasound waves to a subject; and
an ultrasound detecting unit configured to detect, using light, the ultrasound waves reflected off internal tissue of the subject,
wherein said ultrasound detecting unit includes:
a reflector disposed on the subject;
a light source;
an expansion optical system that converts, into planar light, light emitted from said light source and emits substantially parallel light corresponding to the planar light;
a multidivision optical system that divides the planar light resulting from the conversion by said expansion optical system, to form a large number of light-collecting spots on said reflector; and
a light-receiving element that receives reflected light from said reflector.

2. The ultrasound probe according to claim 1,
wherein said ultrasound detecting unit further includes a light-splitting element that splits, into a first light for examination and a second light for reference, the light emitted from said expansion optical system,
the first light forms the light-collecting spots on said reflector,
the first light reflected by said reflector and the second light are multiplexed into the reflected light, and
said light-receiving element receives the reflected light to detect the ultrasound waves reflected off the internal tissue of the subject.

3. The ultrasound probe according to claim 1,
wherein said expansion optical system includes a light guide plate from whose one of main surfaces incident light from an end surface of said light guide plate is emitted substantially vertically, and
said multidivision optical system includes a lens array corresponding to the light-collecting spots.

4. The ultrasound probe according to claim 1, further comprising
a wave guide unit disposed between said ultrasound transmitting unit and the subject or between said ultrasound detecting unit and the subject,
wherein said wave guide unit is configured to deflect the transmitted ultrasound waves or converge the ultrasound waves to be detected.

5. The ultrasound probe according to claim 4,
wherein said wave guide unit includes an ultrasound converging unit configured to converge, for each of a plurality of detection regions, ultrasound waves indicating vibration of a surface of the subject, and
said ultrasound converging unit is configured to propagate, to said reflector, the ultrasound waves indicating the vibration of the surface of the subject, and increase amplitude of the vibration of the ultrasound waves to be propagated to said reflector.

6. The ultrasound probe according to claim 5,
wherein said ultrasound converging unit includes an acoustic lens.

7. The ultrasound probe according to claim 5,
wherein said ultrasound converging unit includes:
a tapered member in form of a protrusion which has a cross-sectional area decreasing in a propagation direction of the ultrasound waves indicating the vibration of the surface of the subject; and
a mirror member located around said tapered member and having an acoustic impedance different from an acoustic impedance of said tapered member, and
the ultrasound waves propagating inside said tapered member propagate to said reflector while being reflected on an interface between said tapered member and said mirror member.

8. The ultrasound probe according to claim 7,
wherein said mirror member includes an air layer.

9. The ultrasound probe according to claim 5,
wherein said reflector includes:
a first multilayer film and a second multilayer film each formed by alternately stacking films having different refractive indices;
a slit for dividing said second multilayer film into the detection regions; and
a spacer formed between said first multilayer film and said second multilayer film, said spacer fixing one end of each of second multilayer films resulting from the division of said second multilayer film by said slit,
said spacer forms a space between said first multilayer film and said second multilayer film, and
said reflector propagates the ultrasound waves converged by said ultrasound converging unit, to an area near the other unfixed end of each of said second multilayer films resulting from the division so that said second multilayer film is deformed, and the propagated ultrasound waves are amplified.

10. The ultrasound probe according to claim 5,
wherein said reflector includes:
a first multilayer film and a second multilayer film each formed by alternately stacking films having different refractive indices;
a slit for dividing said second multilayer film into the detection regions; and
a spacer formed between said first multilayer film and said second multilayer film, said spacer fixing one end of at least every other one of second multilayer films resulting from the division of said second multilayer film by said slit, and
said reflector propagates the ultrasound waves converged by said ultrasound converging unit, to a center or a center of gravity of each of said second multilayer films so that said second multilayer film is deformed, and the propagated ultrasound waves are amplified.

11. The ultrasound probe according to claim 5,
wherein said light source emits the light of a narrow wavelength band,
said reflector includes:
a first multilayer film and a second multilayer film which are each formed by alternately stacking films having different refractive icy indices and have substantially identical reflection properties; and
a substrate on which said first multilayer film is formed and illuminating light emitted from said light source is incident,
said first multilayer film is disposed opposite to said second multilayer film so as to form a resonator structure, and
said reflector propagates the ultrasound waves converged by said ultrasound converging unit, to vary a resonator length of the resonator structure and vary an amount of reflected light originated from the light emitted to the subject.

12. The ultrasound probe according to claim 9,
wherein said ultrasound detecting unit is configured to adjust a wavelength of the light to be emitted from said light source, so as to minimize reflectance in said reflector, and then detect, using the light, the ultrasound waves reflected off the internal tissue of the subject.

13. The ultrasound probe according to claim 4,
wherein said wave guide unit includes an ultrasound deflecting element that deflects the ultrasound waves transmitted by said ultrasound transmitting unit, so as to cause the ultrasound waves to be substantially vertically incident on the subject,
said ultrasound transmitting unit includes a transducer group capable of two-dimensionally or three-dimensionally transmitting the ultrasound waves,
said ultrasound deflecting element is formed integrally with said reflector and is located between said reflector and the subject, and
said reflector is disposed in close contact with the subject via said ultrasound deflecting element.

14. The ultrasound probe according to claim 13,
wherein said ultrasound deflecting element is composed of an acoustic lens.

15. The ultrasound probe according to claim 13,
wherein said ultrasound deflecting element is composed of an acoustic coupler that has a surface inclined with respect to a surface of the subject, and on the inclined surface, said transducer group of said ultrasound transmitting unit is disposed.

16. The ultrasound probe according to claim 13, comprising
a plurality of ultrasound transmitting units each of which is said ultrasound transmitting unit,
wherein said ultrasound deflecting element is formed so that, inside the subject, respective scan ranges of the ultrasound waves from said ultrasound transmitting units have no gap therebetween.

17. The ultrasound probe according to claim 14,
wherein said ultrasound deflecting element is composed of the acoustic lens which includes a combination of transmissive members of two or more kinds having equal optical refractive indices and different sound speeds, and
said acoustic lens is configured so as to refract the ultrasound waves and so as not to refract illuminating light emitted from said light source.

18. An ultrasound examination device comprising:
said ultrasound probe according to claim 1;
a receiving unit configured to amplify and digitally convert a detection signal obtained by demodulating an output signal of said ultrasound probe, and output a signal resulting from the amplification and the digital conversion;
a signal processing unit configured to perform phasing addition using the signal outputted from said receiving unit;
an image processing unit configured to form image data based on data generated by said signal processing unit;
an image display unit configured to display an image based on the image data;
a transmitting unit configured to generate a drive signal for said ultrasound probe to transmit the ultrasound waves; and
a control unit configured to control said transmitting unit so that said transmitting unit generates the drive signal with predetermined timing.

19. The ultrasound examination device according to claim 18, comprising:
an examining unit including said ultrasound transmitting unit and a part of said ultrasound detecting unit and configured to be used in close contact with the subject; and
a main body including at least said signal processing unit, said image processing unit, said image display unit, and said control unit,
wherein said main body includes at least said light source, said light-receiving element, and a part of said optical system.

20. The ultrasound examination device according to claim 18,
wherein said control unit is configured to control an amount of light to be emitted from said light source, according to an elapsed time from the transmission of the ultrasound waves from said ultrasound transmitting unit.
